(19) United States
(12) Patent Application Publication
(10) Pub. No.: US 2021/0212960 A1
(43) Pub. Date: Jul. 15, 2021

(54) IDENTIFICATION OF SEIZURE SUSCEPTIBILITY REGION IN WOLF-HIRSCHHORN SYNDROME AND TREATMENT THEREOF

(71) Applicant: Lineagen, Inc., Salt Lake City, UT (US)

(72) Inventors: Karen S. Ho, Salt Lake City, UT (US); E. Robert Wassman, Salt Lake City, UT (US)

(21) Appl. No.: 17/028,306

(22) Filed: Sep. 22, 2020

Related U.S. Application Data
(63) Continuation of application No. 16/335,234, filed on Mar. 20, 2019, now abandoned.
(60) Provisional application No. 62/397,227, filed on Sep. 20, 2016.

(51) Int. Cl. A61K 31/05; A61P 25/02; A61K 31/4415; A61P 25/08; A61K 31/19; A61K 31/675; C12Q 1/6883

(52) U.S. Cl. CPC A61K 31/05; A61P 25/02; A61K 31/4415; A61P 25/08; C12Q 2600/118; A61K 31/675; C12Q 1/6883; C12Q 2600/106; A61K 31/19

(57) ABSTRACT

The present invention provides methods related to Wolf-Hirschhorn syndrome (WHS), in particular to a 197 kbp chromosomal deletion useful for selecting a patient for anti-seizure therapy (e.g., cannabidiol, vitamin B6, and butyrate), for selecting a particular anti-seizure therapy, and for predicting the response of a subject to a particular anti-seizure therapy.

### CROSS REFERENCE TO RELATED APPLICATIONS

This application is a continuation of U.S. patent application Ser. No. 16/335,234, having a 371(c) date of Mar. 20, 2019, which is the national phase of International Application PCT/US2017/052158 filed Sep. 19, 2017, which claims the benefit of priority from U.S. Provisional Application No. 62/397,227, filed Sep. 20, 2016, each of which is hereby incorporated by reference herein in its entirety.

### FIELD OF THE INVENTION

The present invention relates generally to genetic markers for Wolf-Hirschhorn syndrome (WHS), in particular to a chromosomal deletion for selecting a patient for anti-seizure therapy, for a particular anti-seizure therapy, or predicting the response of a subject to a particular anti-seizure therapy.

### BACKGROUND OF THE INVENTION

Wolf-Hirschhorn syndrome (WHS; OMIM #194190) is a genetic disorder occurring in 1:20,000 to 1:50,000 births (Maas et al. J Med Genet 2008; 45:71-80). Females are approximately twice as likely as males to be affected (Battaglia et al. In: Pagon et al., eds. GeneReviews. Seattle, Wash.: University of Washington, Seattle, 1993. 2015:1-18). The syndrome was first described by Hirschhorn and Cooper in a preliminary report in 1961 and later formalized with back-to-back publications by Wolf et al. and Hirschhorn et al. in Humangenetik in 1965 (Hirschhorn K. Am J Med Genet C Semin Med Genet 2008; 148C:244-5). WHS is characterized by a specific pattern of craniofacial features including a wide nasal bridge that extends to the forehead, widely spaced eyes, distinct mouth, short philtrum, micrognathia, prenatal and postnatal growth delay, intellectual disability (ID) and seizures (Battaglia et al. In: Pagon et al., eds. GeneReviews. Seattle, Wash.: University of Washington, Seattle, 1993. 2015:1-18; Hirschhorn K. Am J Med Genet C Semin Med Genet 2008; 148C:244-5; South et al. Eur J Hum Genet 2008; 16:45-52; Luo et al. Hum Mol Genet 2011; 20:3769-78; Wright et al. Hum Mol Genet 1997; 6:317-24; Lee et al. PLoS One 2014; 9:e106661; Kerzendorfer et al. Hum Mol Genet 2012; 21:2181-93; Endele et al. Genomics 1999; 60:218-25; Rodriguez et al. Am J Med Genet A 2005; 136:175-8; Zollino et al. Am J Hum Genet 2003; 72:590-7; Battaglia et al. Am J Med Genet C Semin Med Genet 2015; 169:216-23). Following identification of these features, WHS has historically been diagnosed by karyotype and/or FISH. Submicroscopic deletions associated with this disorder have more recently been identified by chromosomal microarray analysis (CMA).

Deletions associated with WHS are highly variable in size and genetic content. As a result, individuals with WHS may have additional highly variable clinical features including, e.g., seizures. Epilepsy represents a major clinical challenge during early years, with significant impact on quality of life. Seizures occur in over 90% of individuals with WHS with onset typically within the first 3 years of life and are often induced by low-degree fever (Battaglia et al. Am J Med Genet C Semin Med Genet 2015; 169:216-23).

A significant challenge to understanding the genetics of WHS is the identification of a gene or genes that, when in hemizygous state, give rise to the core features and variable co-morbidities of WHS. Because WHS is a contiguous gene deletion syndrome, loss of one copy of a single gene or the synergistic effects of loss of two or more genes could give rise to the features of WHS. Genotype-phenotype correlation studies of patients with WHS have met with limited success primarily because the prevalence of the disorder is low and therefore assembling a study cohort large enough to achieve statistical power to find significant correlations is difficult; the phenotypic presentation of WHS is highly variable and likely influenced by a number of both genetic and environmental factors; and accurate breakpoint mapping has only become possible within the last decade, and the majority of individuals with a diagnosis of WHS available for such studies have not had CMA as part of their diagnostic workup. The present invention addresses these challenges and more. In particular, the present invention provides insight into the genetics of WHS, including a region of chromosome 4p that, when deleted, is sufficient for seizure occurrence in WHS.

### SUMMARY OF THE INVENTION

The present invention relates to Wolf-Hirschhorn syndrome (WHS), and particularly seizures associated with WHS. More specifically, it relates to a 197 kbp seizure susceptibility region that has been discovered.

One aspect of the invention provides a method for treating WHS seizures comprising administering an effective amount of cannabidiol (CBD) to a subject identified as having a deletion of a seizure susceptibility region, wherein the seizure susceptibility region comprises 197 kbp starting 368 kbp from the terminal end of the short arm of chromosome 4.

In one embodiment, administering CBD reduces the frequency of seizures. In one embodiment, the seizures are one or more of tonic-clonic seizures, clonic seizures, tonic spasms, myoclonic seizures, absence seizures, atonic seizures, complex partial seizures, simple partial seizures, atypical seizures, and status epilepticus. In one embodiment, the deletion of the seizure susceptibility region was detected by chromosomal microarray. In one embodiment, the subject has a diagnosis of WHS. In certain embodiments, the CBD is purified.

Another aspect of the invention provides a method for reducing seizure activity comprising administering an effective amount of cannabidiol (CBD) to a subject identified as having a deletion of a seizure susceptibility region, wherein the seizure susceptibility region comprises 197 kbp starting 368 kbp from the terminal end of the short arm of chromosome 4.

In one embodiment, administering CBD reduces the frequency of seizures. In one embodiment, the seizures are one or more of tonic-clonic seizures, clonic seizures, tonic spasms, myoclonic seizures, absence seizures, atonic seizures, complex partial seizures, simple partial seizures, atypical seizures, and status epilepticus. In one embodiment, the deletion of the seizure susceptibility region was detected by chromosomal microarray. In certain embodiments, the subject has WHS. In one embodiment, the CBD is purified.

One aspect of the invention provides a method for treating Wolf-Hirschhorn syndrome (WHS) seizures comprising administering an effective amount of a combination of vitamin B6 and butyrate to a subject identified as having a deletion of a seizure susceptibility region, wherein the seizure susceptibility region comprises 197 kbp starting 368 kbp from the terminal end of the short arm of chromosome 4.

In one embodiment, administering the combination of vitamin B6 and butyrate reduces the frequency of seizures. In one embodiment, the seizures are one or more of tonic-clonic seizures, clonic seizures, tonic spasms, myoclonic seizures, absence seizures, atonic seizures, complex partial seizures, simple partial seizures, atypical seizures, and status epilepticus. In one embodiment, the deletion of the seizure susceptibility region was detected by chromosomal microarray. In certain embodiments, the subject has a diagnosis of WHS.

Another aspect of the invention provides a method for reducing seizure activity comprising administering a combination of vitamin B6 and butyrate to a subject identified as having a deletion of a seizure susceptibility region, wherein the seizure susceptibility region comprises 197 kbp starting 368 kbp from the terminal end of the short arm of chromosome 4.

In one embodiment, administering the combination of vitamin B6 and butyrate reduces the frequency of seizures. In one embodiment, the seizures are one or more of tonic-clonic seizures, clonic seizures, tonic spasms, myoclonic seizures, absence seizures, atonic seizures, complex partial seizures, simple partial seizures, atypical seizures, and status epilepticus. In one embodiment, the deletion of the seizure susceptibility region was detected by chromosomal microarray. In certain embodiments, the subject has a diagnosis of WHS.

### BRIEF DESCRIPTION OF THE FIGURES

FIG. 1 is a bar graph that shows size and relative locations of 4p deletions of 34 patients with no other clinically reportable CNV findings (henceforth designated as ‘individuals with only 4p deletions’). The deletions of individuals with seizures correspond to patients 1-9, 11-17, 19, 20, 22, 23, and 25-33. Deletions of individuals without seizures correspond to patients 10, 18, 21, 24, and 34. The Wolf-Hirschhorn syndrome (WHS) critical regions 1 and 2 (WHSCR1 and WHSCR2) are shown in black; all patients with the exception of patient 33 have a deletion encompassing both critical regions. Patient 33's deletion partially overlaps with WHSCR2 only and excludes LETM1. Patient 34's deletion starts 751 kbp from the 4p terminus and is the patient deletion that lies closest to the 4p terminus. All chromosome coordinates for this patient group are listed in Table 3.

FIG. 2 is a bar graph that shows the mapping of a candidate seizure propensity region on chromosome 4. Bars show deletion sizes and locations of small 4p terminal or interstitial deletions in the 4p region that help define a 197 kbp seizure susceptibility region. The smallest region of overlap between three patients with seizures is labeled “SEIZURE REGION”. This region is supported by patients from the study cohort (patient numbers labelled on Y-axis) as well as from the literature who have deletions excluding the seizure region and lack seizures (bars filled with diagonal lines indicate no seizures) and patients who have deletions including the seizure region who have seizures (bars filled with cross-hatch indicates a seizure phenotype). Patient data from the literature are indicated along the Y-axis by citation followed by the number of the patient as assigned in the citation in parentheses. Correspondingly, ‘Zollino 2014 (3 and 4)’ labels the size and location of the deletion shared by siblings, patients 3 and 4, in Zollino et al. Epilepsia 2014; 55:849-57. Landmarks such as the Wolf-Hirschhorn syndrome (WHS) critical regions 1 and 2 (WHSCR1 and WHSCR2) are shown, as well as the location of the LETM1 gene. Coordinates are given in base pairs (bps) along the X-axis. Ellipses ( . . . ) indicate that the deletion extends further than shown. Chromosome coordinates for all deletions and regions shown are listed in Table 7.

FIG. 3 is a diagram that shows two genes and a pseudogene lie within the 197 kbp seizure candidate region, PIGG, ZNF721, and pseudogene ABCA11P. The location of this region on Chromosome 4 is shown with the bracket. hg19/GRCh37 coordinates for this region: chr4:367691-564593. The screenshot is from Golden Helix GenomeBrowse® visualization tool V.2.1.0 by GoldenHelix, Inc.

FIG. 4 is a bar graph that shows seizure control outcome by AED.

FIG. 5 is a bar graph that shows seizure control outcome with carbamazepine and levetiracetam.

FIG. 6 is a bar graph that shows seizure control outcome by levetiracetam treatment comparing first use vs. later use.

### DETAILED DESCRIPTION

The present invention relates generally to methods of treating and/or detecting Wolf-Hirschhorn syndrome (WHS), and/or symptoms of WHS, particularly seizures. The present invention also relates, in pertinent part, to the discovery of a 197 kbp seizure susceptibility region (SSR) encompassing two genes and one pseudogene.

In one embodiment, the practice of the present invention will employ, unless indicated specifically to the contrary, conventional methods of virology, immunology, microbiology, molecular biology, bioinformatics, statistics, neurology, pharmacogenetics and pharmacology and recombinant DNA techniques within the skill of the art, many of which are described below for the purpose of illustration. See, e.g., Current Protocols in Molecular Biology or Current Protocols in Immunology, John Wiley & Sons, New York, N.Y. (2009); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used in this specification and the appended claims, the singular forms “a,” “an” and “the” include plural references unless the content clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

As used herein, the term “subject” refers to a vertebrate, for example, a mammal. Thus, the subject can be a human. The term does not denote a particular age or sex. Thus, adult and newborn subjects, as well as fetuses, whether male or female, are intended to be covered. The terms “subject” and “individual” are used interchangeably herein. Unless otherwise specified, the term “patient” includes human and veterinary subjects.

As used herein, the term “biomarker” or “biological marker” means an indicator of a biologic state and may include a characteristic that is objectively measured as an indicator of normal biological processes, pathologic processes, or pharmacologic responses to a therapeutic or other intervention. In one embodiment, a biomarker may indicate a change in expression or state of a protein that correlates with the risk or progression of a disease, or with the susceptibility of the disease in an individual. In certain embodiments, a biomarker may include one or more of the following: genes, proteins, glycoproteins, metabolites, cytokines, and antibodies.

A “copy number variant” (CNV) includes copy number duplications and deletions, and encompasses a copy number change involving a DNA fragment that is about 500 bp or larger (see e.g., Feuk, et al., 2006 Nature Reviews Genetics, 7, 85-97, incorporated by reference in its entirety herein for all purposes). CNVs described herein do not include those variants that arise from the insertion/deletion of transposable elements (e.g., .about.6-kb KpnI repeats) to minimize the complexity of CNV analyses. The term CNV therefore encompasses previously introduced terms such as large-scale copy number variants (LCVs; Iafrate et al. 2004 Nat Genet. 36:949-951, incorporated by reference in its entirety herein for all purposes), copy number polymorphisms (CNPs; Sebat et al. 2004 Science. 305:525-528, incorporated by reference in its entirety herein for all purposes), and intermediate-sized variants (ISVs; Tuzun et al. 2005 Nat Genet. 37:727-732, incorporated by reference in its entirety herein for all purposes), but not retroposon insertions.

With respect to single stranded nucleic acids, particularly oligonucleotides, the term “specifically hybridize” refers to the association between two single-stranded nucleotide molecules of sufficient complementary sequence to permit such hybridization under pre-determined conditions generally used in the art. In particular, in one embodiment the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence. For example, specific hybridization can refer to a sequence which hybridizes to a first chromosomal region but does not specifically hybridize to a second chromosomal region. Appropriate conditions enabling specific hybridization of single stranded nucleic acid molecules of varying complementarity are well known in the art.

The term “oligonucleotide” refers to a relatively short polynucleotide (e.g., 100, 50, 20 or fewer nucleotides) including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

Oligonucleotides of the present invention can be RNA, DNA, or derivatives of either. The minimum size of such oligonucleotides is the size required for formation of a stable hybrid between an oligonucleotide and a complementary sequence on a nucleic acid molecule of the present invention (i.e., the copy number variant genetic markers described herein). The present invention includes oligonucleotides that can be used as, for example, probes to identify nucleic acid molecules (e.g., DNA probes) or primers to amplify nucleic acid molecules.

In one embodiment, an oligonucleotide may be a probe which refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. In certain embodiments, a probe can be between 5 and 100 contiguous bases, and is generally about 5, 10, 12, 13, 14, 15, 16, 17, 18, 19, 20, 21, 22, 23, 24, or 25 nucleotides in length, or may be about 30, 35, 40, 45, 50, 55, 60, 65, 70, 75, 80, 85, 90, 95, or 100 nucleotides in length. The probes herein are selected to be complementary to different strands of a particular target nucleic acid sequence. This means that the probes must be sufficiently complementary so as to be able to specifically hybridize or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

In one embodiment, an oligonucleotide may be a primer, which refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in certain applications, an oligonucleotide primer is about 15-25 or more nucleotides in length, but may in certain embodiments be between 5 and 100 contiguous bases, and often be about 5, 10, 12, 13, 14, 15, 16, 17, 18, 19, 20, 21, 22, 23, 24, or 25 nucleotides long or, in certain embodiments, may be about 30, 35, 40, 45, 50, 55, 60, 65, 70, 75, 80, 85, 90, 95, or 100 nucleotides in length for. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

In the context of the present invention, an “isolated” or “purified” nucleic acid molecule, e.g., a DNA molecule or RNA molecule, is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. In another embodiment, the “isolated nucleic acid” comprises a DNA molecule inserted into a vector, such as a plasmid or virus vector, or integrated into the genomic DNA of a prokaryote or eukaryote. An “isolated nucleic acid molecule” may also comprise a cDNA molecule or an oligonucleotide primer or probe, or additional sequences added onto a fragment of DNA, for example, an adapter sequence added to a restriction cut portion of genomic DNA.

The term “genetic marker” as used herein refers to one or more inherited or de novo variations in DNA structure with a known physical location on a chromosome. Genetic markers include variations, or polymorphisms, in specific nucleotides or chromosome regions. Examples of genetic markers include, single nucleotide polymorphisms (SNPs), and copy number variations and copy number changes (CNVs). Genetic markers can be used to associate an inherited phenotype, such as a disease, with a responsible genotype. Genetic markers may be used to track the inheritance of a nearby gene that has not yet been identified, but whose approximate location is known. The genetic marker itself may be a part of a gene's coding region or regulatory region. For example, a genetic marker may be a functional polymorphism that may alter gene function or gene expression. Alternatively, a genetic marker may be a non-functional polymorphism.

A CNV genetic marker refers to a genomic DNA sequence having a copy number variation, with a known location on a chromosome, which can be used to diagnose subjects with a deletion syndrome, such as WHS and/or to select a subject for treatment of such a syndrome.

A single nucleotide polymorphism (SNP) refers to a change in which a single base in the DNA differs from the usual base at that position. These single base changes are called SNPs or “snips.” Millions of SNPs have been cataloged in the human genome. Some SNPs such as that which causes sickle cell are responsible for disease. Other SNPs are normal variations in the genome.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, in one embodiment, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

“Sample” or “biological sample,” as used herein, refers to a sample obtained from a human subject or a patient, which may be tested for a particular molecule, for example one or more of the CNVs associated with a deletion or duplication syndrome, as set forth herein. Samples may include but are not limited to cells, buccal swab sample, body fluids, including blood, serum, plasma, urine, saliva, cerebral spinal fluid, tears, pleural fluid and the like. Samples that are suitable for use in the methods described herein contain genetic material, e.g., genomic DNA (gDNA). Non-limiting examples of sources of samples include urine, blood, and tissue. The sample itself will typically consist of nucleated cells (e.g., blood or buccal cells), tissue, etc., removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

### Wolf-Hirschhorn Syndrome

Wolf-Hirschhorn syndrome (WHS) is a contiguous gene deletion syndrome involving variable size deletions of the 4p16.3 region. WHS is characterized by a specific pattern of craniofacial features including a wide nasal bridge that extends to the forehead, widely spaced eyes, distinct mouth, short philtrum, micrognathia, prenatal and postnatal growth delay, intellectual disability (ID) and seizures (Battaglia et al. In: Pagon et al., eds. GeneReviews. Seattle, Wash.: University of Washington, Seattle, 1993. 2015:1-18; Hirschhorn K. Am J Med Genet C Semin Med Genet 2008; 148C:244-5; South et al. Eur J Hum Genet 2008; 16:45-52; Luo et al. Hum Mol Genet 2011; 20:3769-78; Wright et al. Hum Mol Genet 1997; 6:317-24; Lee et al. PLoS One 2014; 9:e106661; Kerzendorfer et al. Hum Mol Genet 2012; 21:2181-93; Endele et al. Genomics 1999; 60:218-25; Rodriguez et al. Am J Med Genet A 2005; 136:175-8; Zollino et al. Am J Hum Genet 2003; 72:590-7; Battaglia et al. Am J Med Genet C Semin Med Genet 2015; 169:216-23). Following identification of these features, WHS has historically been diagnosed by karyotype and/or FISH. Submicroscopic deletions associated with this disorder have more recently been identified by chromosomal microarray analysis (CMA).

In addition to the core features of WHS listed above, additional highly variable clinical features of WHS include, but are not limited to, feeding difficulties, congenital heart defects, hearing loss, skeletal anomalies, kidney and urinary tract malformations, and ophthalmological and dental abnormalities (Battaglia et al. In: Pagon et al., eds. GeneReviews. Seattle, Wash.: University of Washington, Seattle, 1993. 2015:1-18). Terminal deletion resulting in partial monosomy of chromosome 4p is the most common cause of WHS. Interstitial deletions, unbalanced translocations, ring chromosomes and other complex genetic rearrangements can also give rise to WHS (Battaglia et al. In: Pagon et al., eds. GeneReviews. Seattle, Wash.: University of Washington, Seattle, 1993. 2015:1-18; South et al. Eur J Hum Genet 2008; 16:45-52; Luo et al. Hum Mol Genet 2011; 20:3769-78). As a result, deletions associated with WHS are highly variable in size and genetic content, potentially causing or contributing to the variability in presentation of this disorder.

Two adjacent regions, located approximately 1.8-2.0 Mbp from the 4p terminus, are each proposed to be the minimal region of deletion necessary to observe the core WHS features. These regions were identified based on determination of the smallest region of overlap (SRO) of individuals with WHS. The first critical region described was a 165 kbp interval encompassing part of the WHSC1 gene and all of the WHSC2 (NELFA) gene (Wright et al. Hum Mol Genet 1997; 6:317-24). These genes play a role in the regulation of key bone differentiation genes (Lee et al. PLoS One 2014; 9:e106661) and regulation of DNA replication and cell-cycle progression (Kerzendorfer et al. Hum Mol Genet 2012; 21:2181-93).

Chromosomal deletion syndromes, such as WHS, are often associated with developmental delay. WO 2015/157571 provides methods for determining whether a subject's genomic DNA includes a copy number variant (“CNV”) at one or more chromosomal locations associated with a deletion syndrome, such as WHS. The disclosure of WO 2015/157571 is incorporated by reference herein in its entirety.

### Seizure Susceptibility Region

Seizures are frequently, but not always, associated with WHS. Epilepsy represents a major clinical challenge during early years, with significant impact on quality of life. Seizures occur in over 90% of individuals with WHS with onset typically within the first 3 years of life and are often induced by low-degree fever (Battaglia et al. Am J Med Genet C Semin Med Genet 2015; 169:216-23). The most frequently occurring seizure types are generalized tonic-clonic seizures, tonic spasms, complex partial seizures and clonic seizures. Unilateral/generalized clonic or tonic-clonic status epilepticus occurs in 50% of individuals with WHS (Battaglia et al. Am J Med Genet C Semin Med Genet 2015; 169:216-23).

As described in Example 1 below, the inventors have identified a 197 kbp seizure susceptibility region. Deletion of the seizure susceptibility region is sufficient for seizure activity in WHS individuals. The seizure susceptibility region is the 197 kbp region starting 368 kbp from the terminal end of chromosome 4. The 197 kbp seizure susceptibility region encompasses two genes, ZNF721 and PIGG, and one pseudogene, ABCA11P. ZNF721 encodes a zinc-finger-containing protein of unknown function, PIGG encodes a member of the phosphatidylinositol glycan anchor biosynthetic pathway and ABCA11P is a pseudogene with sequence similarity to ATP-binding cassette, subfamily A.

In one embodiment, a subject has a deletion of a 197 kbp seizure susceptibility region starting 368 kbp from the terminal end of the short arm of chromosome 4. In one embodiment, the deletion comprises the entire 197 kbp. In one embodiment, the deletion comprises a part of the seizure susceptibility region. In one embodiment, the part of the seizure susceptibility region comprises the PIGG gene.

In one embodiment, the deletion of the seizure susceptibility region is part of a larger CNV deletion. For example, in one embodiment the size of the deletion is at least about 500 bp, at least about 1,000 bp, at least about 10,000 bp, at least about 100,000 bp, at least about 1 mega base pairs (Mb), at least about 5 Mb, at least about 10 Mb, at least about 15 Mb, at least about 20 Mb, or at least about 50 Mb. CNVs and their respective size are detected by nucleic acid hybridization assays with primers (oligonucleotides) that specifically hybridize to the chromosomal DNA of interest.

One embodiment of the invention provides a method for detecting a deletion of the 197 kbp seizure susceptibility region in an individual having, or suspected of having, WHS. In one embodiment, deletion of the 197 kbp seizure susceptibility region indicates the individual is predisposed, or likely, to have seizures.

Deletion of the seizure susceptibility region may be detected by a variety of methods known in the art, e.g., a DNA hybridization assay. Once a sample is obtained, it is interrogated for deletion of the seizure susceptibility region, e.g., within a CNV.

In one embodiment, the deletion of the seizure susceptibility region is part of a CNV. The method in one embodiment comprises probing a sample obtained from the subject for the presence or absence of one or more CNVs associated with WHS, and if the CNV is present, optionally analyzing the size of the deletion of at least one CNV. In one embodiment, the probing step comprises mixing the sample with five or more oligonucleotides that are substantially complementary to portions of the genomic DNA sequence associated with the deletion syndrome under conditions suitable for hybridization of the five or more oligonucleotides to their complements or substantial complements; detecting whether hybridization occurs between the five or more oligonucleotides to their complements or substantial complements, or a subset thereof and obtaining hybridization values of the sample based on the detecting step.

The determination of whether the seizure susceptibility region is present or absent, in one embodiment, comprises comparing the hybridization values of the sample to reference hybridization value(s) from at least one training set comprising hybridization value(s) from a sample that is positive for the seizure susceptibility region, or hybridization value(s) from a sample that is negative for the seizure susceptibility region. In one embodiment, the comparing step comprises determining a correlation between the hybridization values obtained from the sample and the hybridization value(s) from the at least one training set (which may be included in a database of values or a sample training set). A determination is then made regarding the presence or absence of the seizure susceptibility region.

In one embodiment, the sample comprises restriction digested double stranded DNA obtained from genomic DNA fragments; restriction digested single stranded DNA obtained from genomic DNA fragments; amplified restriction digested genomic DNA single stranded fragments; amplified restriction digested genomic DNA double stranded fragments; or a combination thereof. In a further embodiment, the sample is free of histone proteins. In even a further embodiment, the amplified restriction digested genomic DNA single stranded fragments comprise a detectable label chemically attached to individual single stranded fragments. In yet a further embodiment, the amplified restriction digested genomic DNA single stranded fragments further comprise adapter sequences. In one embodiment, the adapter sequences are introduced via adapter-specific primers.

In each of the methods described herein, the presence or absence of the seizure susceptibility region described herein is probed for in a sample obtained from a subject. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., genomic DNA. All samples obtained from a subject, including those subjected to any sort of further processing, are considered to be obtained from the subject.

The sample in one embodiment, is further processed before the detection of the presence or absence of the seizure susceptibility region. For example, DNA, e.g., genomic DNA in a cell or tissue sample can be separated from other components of the sample. The sample can be concentrated and/or purified to isolate genomic DNA in a non-natural state. Specifically, genomic DNA exists as genomic chromosomal DNA and is a tightly coiled structure, wherein the DNA is coiled many times around histone proteins that support the genomic DNA and chromosomal structure. In the methods provided herein, the higher order structure of the genomic DNA (e.g., tertiary and quaternary structures) is modified considerably by eliminating histone proteins from the sample, and digesting the genomic DNA into fragments with frequent cutting restriction endonucleases. Genomic DNA therefore does not exist as natural genomic DNA, it is present in small fragments (with lengths ranging from about 100 base pairs to about 500 base pairs) rather than as large polymers on individual chromosomes, comprising tens to hundreds of megabase pairs.

Once the genomic DNA is digested and chemically modified into a non-natural sequence and structure, it is amplified, in one embodiment, with primers that introduce an additional DNA sequence (adapter sequence) onto the fragments (with the use of adapter-specific primers). Amplification therefore serves to create non-natural double stranded molecules, by introducing adapter sequences into the already non-natural restriction digested, and chemically modified genomic DNA. Further, as known to those of ordinary skill in the art, amplification procedures have error rates associated with them. Therefore, amplification introduces further modifications into the smaller DNA fragments. In one embodiment, during amplification with the adapter-specific primers, a detectable label, e.g., a fluorophore, is added to single strand DNA fragments. Amplification therefore also serves to create DNA complexes that do not occur in nature, at least because of (i) the addition of adapter sequences, (ii) the error rate associated with amplification, (iii) the disparate structure of these complexes as compared to what exists in nature, i.e., large polymers of DNA wrapped around histone proteins and the chemical addition of a detectable label to the DNA fragments.

In general, the seizure susceptibility region can be identified using a nucleic acid hybridization assay alone or in combination with an amplification assay, i.e., to amplify the nucleic acid in the sample prior to detection. In one embodiment, the genomic DNA of the sample is sequenced or hybridized to an array, as described in detail herein. A determination is then made as to whether the sample includes the seizure susceptibility region depending on the detected hybridization pattern, or rather, includes the “normal” or “wild type” sequence (also referred to as a “reference sequence” or “reference allele”).

Detection using a hybridization assay comprises the generation of non-natural DNA complexes, that is, DNA complexes that do not exist in nature. As mentioned above, the DNA that is used in the hybridization assay is already in a non-natural state because of various modifications, specifically, (i) modifications to the length of the DNA, (ii) modifications to the primary structure of the DNA via the addition of adapter sequences during the amplification process, (iii) modifications to the higher order structure of the DNA due to the elimination of histone proteins and other cellular material, (iv) chemical modifications due to the addition of a detectable label to the digested DNA fragments, and (v) further chemical modifications due to introduction of bases that do not occur in the native chromosomal DNA, due to inherent error in the amplification reaction (leading to further change in primary structure as compared to chromosomal genomic DNA).

In the case of a hybridization assay, for example a microarray assay or bead based assay, hybridization occurs between the non-natural fragments described above and an immobilized sequence of known identity. Therefore, the product of the hybridization assay is further removed from DNA duplexes that exist in nature, because of the reasons set forth above, and because each is immobilized, for example to a glass slide or bead.

In one embodiment, if the hybridization assay reveals a difference between the sequenced region and the reference sequence (which can be included in the hybridization assay as a control, or in a dataset, for example, a statistical training set), a chromosomal deletion (e.g., CNV) has been identified. Certain statistical algorithms can aid in this determination, as described herein. The fact that a difference in nucleotide sequence is identified at a particular site that determines that a CNV exists at that site.

For example, an oligonucleotide or oligonucleotide pair can be used in the methods described herein, for example in a microarray (e.g., CMA) or polymerase chain reaction assay, to detect the one or more seizure susceptibility region-containing CNVs. As used herein a set of oligonucleotides, in one embodiment, comprises from about 2 to about 100 oligonucleotides, all of which specifically hybridize to a particular CNV or region thereof. In one embodiment, a set of oligonucleotides comprises from about 5 to about 100 oligonucleotides (or from about 5 to about 30 oligonucleotide pairs), from about 10 to about 100 oligonucleotides (or from about 10 to about 100 oligonucleotide pairs), from about 10 to about 75 oligonucleotides (or from about 10 to about 75 oligonucleotide pairs), from about 10 to about 50 oligonucleotides (or from about 10 to about 0 oligonucleotide pairs). In one embodiment, a set of oligonucleotides comprises about 15 to about 50 oligonucleotides, all of which specifically hybridize to a particular CNV associated with WHS. In one embodiment, a set of oligonucleotides comprises DNA probes, e.g., genomic DNA probes. In one embodiment, the DNA probes comprise DNA probes that overlap in genomic sequence. In another embodiment, the DNA probes comprise DNA probes that do not overlap in genomic sequence. In one embodiment, the DNA probes provide detection coverage over the length of a CNV associated with WHS. In another embodiment, a set of oligonucleotides comprises amplification primers that amplify a CNV or region thereof, wherein the CNV is associated with WHS. In this regard, sets of oligonucleotides comprising amplification primers may comprise multiplex amplification primers. In another embodiment, the sets of oligonucleotides or DNA probes may be provided on an array, such as solid phase arrays, chromosomal/DNA microarrays, or micro-bead arrays.

In one embodiment, an array for identifying the genotype of a subject suspected of having WHS, comprises the DNA probes set forth in Table 14 from WO 2014/055915, the disclosure of which is incorporated by reference in its entirety. For example, in one embodiment, detecting a deletion of the 197 kbp seizure susceptibility region comprises chromosomal microarray (CMA) analysis. In one embodiment, the CMA is FirstStepDX PLUS® (Lineagen). Exemplary nucleotide probes that may be used to detect a deletion of the 197 kbp seizure susceptibility region include, but are not limited to, the probes listed in Table 1. The probes are 25 nucleotides in length, and the central nucleotide is listed as the genomic coordinate.

TABLE 1
Exemplary nucleotide probes

| Chromosome | Coordinate (hg19) | Probe Name |
|---|---|---|
| chr4 | 367740 | C-4TSKB |
| chr4 | 369687 | C-3ANEA |
| chr4 | 369953 | C-4BOFD |
| chr4 | 370019 | C-4OPJX |
| chr4 | 370152 | C-4TVPN |
| chr4 | 370579 | C-6DPHJ |
| chr4 | 370645 | C-4UHZW |
| chr4 | 370694 | C-7FYGI |
| chr4 | 370970 | C-5SKEZ |
| chr4 | 371055 | C-4KZSD |
| chr4 | 371139 | C-5QYKC |
| chr4 | 371301 | C-5NYSI |
| chr4 | 371321 | C-4TEOP |
| chr4 | 371336 | C-5ESYZ |
| chr4 | 373977 | C-6QDYP |
| chr4 | 374016 | C-3QQXO |
| chr4 | 374030 | C-6HDZJ |
| chr4 | 378278 | C-7RPOQ |
| chr4 | 378398 | C-4YSTU |
| chr4 | 386073 | C-7BVNV |
| chr4 | 386745 | C-4KUIM |
| chr4 | 387096 | C-4NMKJ |
| chr4 | 390582 | C-7PZWV |
| chr4 | 390613 | C-4BEKN |
| chr4 | 390643 | C-4ITPK |
| chr4 | 390644 | C-3TAHB |
| chr4 | 390658 | C-6GCEL |
| chr4 | 390673 | C-3KRYK |
| chr4 | 390692 | C-7IWMY |
| chr4 | 390743 | C-4BLIL |
| chr4 | 394184 | S-3GIKU |
| chr4 | 394588 | C-4LYEC |
| chr4 | 396936 | C-7PWXU |
| chr4 | 397109 | C-6IIJR |
| chr4 | 406363 | C-5LKXW |
| chr4 | 407055 | C-6YWUG |
| chr4 | 407056 | C-6VSBC |
| chr4 | 407071 | C-6LZER |
| chr4 | 407072 | C-5ZISO |
| chr4 | 418084 | C-6DPFF |
| chr4 | 418131 | C-6GAHD |
| chr4 | 418132 | C-3ACKR |
| chr4 | 418191 | C-3VYFP |
| chr4 | 418244 | C-7RJMH |
| chr4 | 418834 | S-3YTPG |
| chr4 | 419601 | C-6ROPT |
| chr4 | 419613 | C-7AYPQ |
| chr4 | 419680 | C-6MSWV |
| chr4 | 419888 | C-6RTZI |
| chr4 | 419924 | C-5UYTD |
| chr4 | 419925 | C-3KWSZ |
| chr4 | 421086 | S-3CHTP |
| chr4 | 421584 | C-5ANWV |
| chr4 | 421621 | S-4TBKU |
| chr4 | 422001 | C-4CGTJ |
| chr4 | 422497 | C-4RXHF |
| chr4 | 422535 | C-6PJWK |
| chr4 | 422536 | C-5QISF |
| chr4 | 422822 | C-5QFHE |
| chr4 | 422823 | C-4WMKH |
| chr4 | 429021 | C-5WVFZ |
| chr4 | 429091 | C-5XLJP |
| chr4 | 429092 | C-5WHWX |
| chr4 | 429358 | C-7KJHO |
| chr4 | 429611 | C-3YWMK |
| chr4 | 429646 | C-5YOUR |
| chr4 | 429647 | C-4VDJA |
| chr4 | 429720 | S-4GUKQ |
| chr4 | 429749 | C-7EJIH |
| chr4 | 430221 | S-3YNZL |
| chr4 | 432586 | C-5CMBD |
| chr4 | 432600 | C-5KMIC |
| chr4 | 432628 | C-5NCNL |
| chr4 | 433216 | C-3XVLV |
| chr4 | 433694 | C-4BQPD |
| chr4 | 433756 | C-5AAHX |
| chr4 | 433911 | C-7AAJW |
| chr4 | 433956 | C-4JDSU |
| chr4 | 434007 | C-6CXGZ |
| chr4 | 434079 | S-4SKHJ |
| chr4 | 434088 | C-4DVAG |
| chr4 | 436370 | C-7NOCJ |
| chr4 | 436459 | C-5CQQX |
| chr4 | 436544 | C-5RMJQ |
| chr4 | 436760 | C-3CXGY |
| chr4 | 436812 | C-7DUQJ |
| chr4 | 436862 | C-7BMGB |
| chr4 | 436963 | C-5TGHL |
| chr4 | 437132 | C-7CGHX |
| chr4 | 437148 | C-4YDFX |
| chr4 | 437216 | C-5SQXG |
| chr4 | 438067 | S-4CGMC |
| chr4 | 438414 | C-6AWOC |
| chr4 | 438538 | S-4HKWG |
| chr4 | 438626 | C-4IHBF |
| chr4 | 438663 | S-3MQCB |
| chr4 | 439074 | C-5OAAK |
| chr4 | 439075 | C-4ORKX |
| chr4 | 439538 | C-4SSXY |
| chr4 | 439570 | C-6CHAU |
| chr4 | 439571 | C-4PCHD |
| chr4 | 439626 | C-3OUNE |
| chr4 | 439677 | C-5LDHG |
| chr4 | 440693 | C-4WHEE |
| chr4 | 440762 | C-5BVLH |
| chr4 | 443645 | C-6VCXH |
| chr4 | 443735 | C-3GEBJ |
| chr4 | 443817 | C-7MUUQ |
| chr4 | 443833 | C-5SBYP |
| chr4 | 443834 | C-4YWSL |
| chr4 | 444267 | C-6QTLC |
| chr4 | 444283 | C-5IOQZ |
| chr4 | 444284 | C-4QYME |
| chr4 | 444378 | C-7FCSE |
| chr4 | 446553 | C-3KPZZ |
| chr4 | 446910 | C-6OILV |
| chr4 | 446924 | C-3QBVN |
| chr4 | 447157 | C-3PIYA |
| chr4 | 447505 | C-4NHOU |
| chr4 | 447730 | C-7CZLE |
| chr4 | 451082 | C-3BYSC |
| chr4 | 451105 | C-4SIRL |
| chr4 | 451119 | C-3UBVU |
| chr4 | 451252 | C-5NZQA |
| chr4 | 451510 | C-4TYCD |
| chr4 | 451532 | C-3LHFV |
| chr4 | 451631 | C-6VPYL |
| chr4 | 452071 | C-4QIDA |
| chr4 | 452072 | C-4BEOQ |
| chr4 | 452351 | S-3VUQL |
| chr4 | 452926 | C-3ZKYB |
| chr4 | 452962 | C-4WOMB |
| chr4 | 462416 | S-4DQKO |
| chr4 | 462656 | C-4MGET |
| chr4 | 462786 | C-3XHKY |
| chr4 | 462801 | C-4PGEH |
| chr4 | 462836 | C-5JBXH |
| chr4 | 462848 | C-6FNDY |
| chr4 | 463298 | C-5RPJJ |
| chr4 | 463299 | C-3VWUZ |
| chr4 | 463312 | C-5HYEO |
| chr4 | 463394 | C-3VSPO |
| chr4 | 463409 | C-4TSHZ |
| chr4 | 463554 | C-7OSNR |
| chr4 | 463961 | C-5HCOA |
| chr4 | 463962 | C-3ZMEW |
| chr4 | 471957 | C-5ZRSL |
| chr4 | 472000 | S-3TAYN |
| chr4 | 472016 | C-3NVVK |
| chr4 | 473635 | C-4XHRA |
| chr4 | 473898 | S-3PTOQ |
| chr4 | 473981 | C-5OGXJ |
| chr4 | 474034 | C-4DPJS |
| chr4 | 474075 | C-6BHWQ |
| chr4 | 474142 | C-6WXWY |
| chr4 | 474269 | C-6CQBN |
| chr4 | 474435 | C-3ZTWF |
| chr4 | 475468 | S-3GPGO |
| chr4 | 476370 | C-4MAIG |
| chr4 | 476617 | C-3XYOL |
| chr4 | 476632 | C-3XAXT |
| chr4 | 476949 | C-5ZKJV |
| chr4 | 477429 | C-3ZDBP |
| chr4 | 477737 | C-5IFSH |
| chr4 | 477801 | C-4JODM |
| chr4 | 478314 | C-6BVLT |
| chr4 | 478554 | C-6RJRE |
| chr4 | 481423 | C-4TWRP |
| chr4 | 481796 | C-7JLRX |
| chr4 | 482054 | C-4JGWN |
| chr4 | 482111 | C-5TFMY |
| chr4 | 482823 | C-6SNNA |
| chr4 | 482950 | C-4MQOE |
| chr4 | 483307 | C-5KAAO |
| chr4 | 483408 | C-5YDDE |
| chr4 | 483521 | C-6HDOL |
| chr4 | 483536 | C-5AMOM |
| chr4 | 484395 | C-4VXCN |
| chr4 | 484528 | C-4SNXA |
| chr4 | 484803 | C-3OKHY |
| chr4 | 484818 | C-3FZJA |
| chr4 | 484884 | C-6YYTT |
| chr4 | 485233 | C-3XYWT |
| chr4 | 485289 | C-6DKXW |
| chr4 | 490074 | S-3XZYX |
| chr4 | 495493 | C-3WVZV |
| chr4 | 495647 | S-3GOIB |
| chr4 | 495682 | C-7LEWE |
| chr4 | 495683 | C-3LEOB |
| chr4 | 495956 | C-3FPRD |
| chr4 | 496243 | C-5RGIU |
| chr4 | 497138 | S-4NEPB |
| chr4 | 500522 | C-7ANDE |
| chr4 | 500523 | C-5RGUE |
| chr4 | 500585 | C-7HKCO |
| chr4 | 500631 | S-4EMSX |
| chr4 | 501423 | C-6WRNN |
| chr4 | 501424 | C-4PFWL |
| chr4 | 502732 | C-4RSNK |
| chr4 | 502844 | C-5QOKT |
| chr4 | 502911 | S-3MIBQ |
| chr4 | 502913 | C-4CYXB |
| chr4 | 502987 | C-4RLMX |
| chr4 | 502999 | C-6IPIB |
| chr4 | 503018 | C-5RLQR |
| chr4 | 504410 | C-4LONS |
| chr4 | 505201 | C-5YPGF |
| chr4 | 505604 | C-4ANKC |
| chr4 | 507338 | C-6APRW |
| chr4 | 507662 | C-7LHYM |
| chr4 | 507663 | C-3YMUC |
| chr4 | 512828 | S-4ONMR |
| chr4 | 513115 | C-6USWM |
| chr4 | 513116 | C-3DTFN |
| chr4 | 513127 | C-3MTGZ |
| chr4 | 513414 | S-3BNNS |
| chr4 | 517198 | S-3XFJA |
| chr4 | 517199 | C-6HHWG |
| chr4 | 517376 | S-4CTDX |
| chr4 | 517636 | C-6GMRG |
| chr4 | 517735 | C-3CMYH |
| chr4 | 521896 | C-5XKSP |
| chr4 | 521942 | C-6CAIY |
| chr4 | 522403 | C-4ZPYO |
| chr4 | 522527 | C-4LFJC |
| chr4 | 522743 | S-4OCJI |
| chr4 | 523768 | C-5OBKH |
| chr4 | 523786 | C-5UQOH |
| chr4 | 523801 | C-3IREN |
| chr4 | 523849 | C-3QQMZ |
| chr4 | 525310 | C-3GPSD |
| chr4 | 525981 | C-6EHNY |
| chr4 | 526241 | C-6BOMQ |
| chr4 | 526306 | S-4QHOA |
| chr4 | 528289 | S-3VUUC |
| chr4 | 528365 | C-5CUNF |
| chr4 | 528694 | C-6DSML |
| chr4 | 528944 | C-4XBOX |
| chr4 | 528981 | C-7JBVP |
| chr4 | 529301 | C-4JDSY |
| chr4 | 531969 | C-5ASWS |
| chr4 | 532532 | C-6GYHE |
| chr4 | 532560 | C-7NXCR |
| chr4 | 532966 | C-7HWOA |
| chr4 | 533001 | S-3XTZL |
| chr4 | 533002 | C-3MWYH |
| chr4 | 533110 | C-3TPZU |
| chr4 | 533256 | C-4KSYD |
| chr4 | 533342 | C-3UIAD |
| chr4 | 533399 | S-3UOAE |
| chr4 | 533400 | C-6KJTC |
| chr4 | 533541 | C-7DGJD |
| chr4 | 540025 | S-4CHUS |
| chr4 | 540099 | S-4MERC |
| chr4 | 540145 | C-6BEVR |
| chr4 | 540836 | C-3TOIM |
| chr4 | 541182 | S-3LTWI |
| chr4 | 541595 | S-3MLIE |
| chr4 | 541714 | C-5JCFO |
| chr4 | 541715 | C-3GZXB |
| chr4 | 541995 | C-5IJLJ |
| chr4 | 542015 | C-5MNSC |
| chr4 | 542016 | C-3BPBX |
| chr4 | 542090 | C-5ESTM |
| chr4 | 548603 | C-4VDBK |
| chr4 | 548722 | C-4XMIJ |
| chr4 | 552700 | C-5BRUE |
| chr4 | 552736 | C-4AQSF |
| chr4 | 552862 | S-4AXPP |
| chr4 | 553718 | C-3IJJV |
| chr4 | 553918 | C-3PJBO |
| chr4 | 554266 | C-6UIQR |
| chr4 | 554277 | C-7PIEE |
| chr4 | 554543 | S-3ZIKM |
| chr4 | 554586 | C-5NCNB |
| chr4 | 554620 | C-6FFSG |
| chr4 | 554710 | S-3CUUD |
| chr4 | 561923 | C-5PWDY |
| chr4 | 563192 | S-3FYSN |
| chr4 | 563744 | C-5PLRK |
| chr4 | 563856 | C-6GRUN |

In one embodiment, hybridization on a microarray is used to detect the presence of one or more SNPs in a patient's sample. The term “microarray” refers to an ordered arrangement of hybridizable array elements, e.g., polynucleotide probes, on a substrate.

In another embodiment of the invention, constant denaturant capillary electrophoresis (CDCE) can be combined with high-fidelity PCR (HiFi-PCR) to detect the presence of one or more CNVs. In another embodiment, high-fidelity PCR is used. In yet another embodiment, denaturing HPLC, denaturing capillary electrophoresis, cycling temperature capillary electrophoresis, allele-specific PCRs, quantitative real time PCR approaches such as TaqMan® is employed to detect the one or more CNVs. Other approaches to detect the presence of one or more CNVs, and in some cases, the size (i.e., as reported in bases or base pairs) of the one or more CNVs, amenable for use with the present invention include polony sequencing approaches, microarray approaches, mass spectrometry, high-throughput sequencing approaches, e.g., at a single molecule level, and the NanoString approach.

Hybridization detection methods are based on the formation of specific hybrids between complementary nucleic acid sequences that serve to detect nucleic acid sequence mutation(s) and are amenable for use with the methods described herein. Methods of nucleic acid analysis to detect polymorphisms and/or polymorphic variants (copy number variants) include, e.g., microarray analysis and real time PCR. Hybridization methods, such as Southern analysis, Northern analysis, or in situ hybridizations, can also be used (see Current Protocols in Molecular Biology, Ausubel et al., eds., John Wiley & Sons 2003, incorporated by reference in its entirety).

Other methods for use with the methods provided herein include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al. U.S. Pat. No. 5,288,644, each incorporated by reference in its entirety for all purposes); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CS GE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989), incorporated by reference in its entirety), restriction enzyme analysis (Flavell et al., Cell 15:25 (1978); Geever et al., Proc. Natl. Acad. Sci. USA 78:5081 (1981), incorporated by reference in its entirety); quantitative real-time PCR (Raca et al., Genet Test 8(4):387-94 (2004), incorporated by reference in its entirety); heteroduplex analysis; chemical mismatch cleavage (CMC) (Cotton et al., Proc. Natl. Acad. Sci. USA 85:4397-4401 (1985), incorporated by reference in its entirety); RNase protection assays (Myers et al., Science 230:1242 (1985), incorporated by reference in its entirety); use of polypeptides that recognize nucleotide mismatches, e.g., E. coli mutS protein; allele-specific PCR, for example. See, e.g., U.S. Patent Publication No. 2004/0014095, which is incorporated herein by reference in its entirety.

In order to detect the seizure susceptibility region described herein, in one embodiment, genomic DNA (gDNA) or a portion thereof containing the polymorphic site, present in the sample obtained from the subject, is first amplified. Such regions can be amplified and isolated by PCR using oligonucleotide primers designed based on genomic and/or cDNA sequences that flank the site. See e.g., PCR Primer: A Laboratory Manual, Dieffenbach and Dveksler, (Eds.); McPherson et al., PCR Basics: From Background to Bench (Springer Verlag, 2000, incorporated by reference in its entirety); Mattila et al., Nucleic Acids Res., 19:4967 (1991), incorporated by reference in its entirety; Eckert et al., PCR Methods and Applications, 1:17 (1991), incorporated by reference in its entirety; PCR (eds. McPherson et al., IRL Press, Oxford), incorporated by reference in its entirety; and U.S. Pat. No. 4,683,202, incorporated by reference in its entirety. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), incorporated by reference in its entirety, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety. A variety of computer programs for designing primers are available.

In one example, a sample (e.g., a sample comprising genomic DNA), is obtained from a subject. The DNA in the sample is then examined to determine a chromosomal deletion profile as described herein. The profile is determined by any method described herein, e.g., by sequencing or by hybridization of genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In certain embodiments, the oligonucleotides for detecting a deletion of the seizure susceptibility region may be used in high throughput sequencing methods (often referred to as next-generation sequencing methods or next-gen sequencing methods). Accordingly, in one embodiment, the present disclosure provides methods of determining or predicting the presence or absence of a deletion by detecting in a genetic sample from the subject one or more CNVs by high throughput sequencing. High throughput sequencing, or next-generation sequencing, methods are known in the art (see, e.g., Zhang et al., J Genet Genomics. 2011 Mar. 20; 38(3):95-109; Metzker, Nat Rev Genet. 2010 Jan.; 11(1):31-46, incorporated by reference herein in its entirety) and include, but are not limited to, technologies such as ABI SOLiD sequencing technology (now owned by Life Technologies, Carlsbad, Calif.); Roche 454 FLX which uses sequencing by synthesis technology known as pyrosequencing (Roche, Basel Switzerland); Illumina Genome Analyzer (Illumina, San Diego, Calif.); Dover Systems Polonator G.007 (Salem, N.H.); Helicos (Helicos BioSciences Corporation, Cambridge Mass., USA), and Sanger. In one embodiment, DNA sequencing may be performed using methods well known in the art including mass spectrometry technology and whole genome sequencing technologies (e.g., those used by Pacific Biosciences, Menlo Park, Calif., USA), etc.

In one embodiment, nucleic acid, for example, genomic DNA is sequenced using nanopore sequencing, to determine the presence of a deletion of the seizure susceptibility region (e.g., as described in Soni et al. (2007). Clin Chem 53, pp. 1996-2001, incorporated by reference in its entirety for all purposes). Nanopore sequencing is a single-molecule sequencing technology whereby a single molecule of DNA is sequenced directly as it passes through a nanopore. A nanopore has a diameter on the order of 1 nanometer. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. patent application publications U.S. Patent Application Publication Nos. 2006/003171 and 2009/0029477, each incorporated by reference in its entirety for all purposes, is amenable for use with the methods described herein

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence. The probe sequence must also be sufficiently long so that the probe exhibits selectivity for the target sequence over non-target sequences. For example, the probe will be at least 10, e.g., 15, 20, 25, 30, 35, 50, 100, or more, nucleotides in length. In some embodiments, the probes are not more than 30, 50, 100, 200, 300, or 500 nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

Control probes can also be used. For example, a probe that binds a less variable sequence, e.g., repetitive DNA associated with a centromere of a chromosome, or a probe that exhibits differential binding to the polymorphic site being interrogated, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK).

In some embodiments, the probes are labeled with a detectable label, e.g., by direct labeling. In various embodiments, the oligonucleotides for detecting the seizure susceptibility region described herein are conjugated to a detectable label that may be detected directly or indirectly. In the present invention, oligonucleotides may all be covalently linked to a detectable label.

In one embodiment, CNV size is determined via a nucleic acid hybridization method as follows. Oligonucleotide probes are employed and each represents a known chromosomal coordinate based on hg19 coordinates. In a subject who has no deletion or duplication in a particular region, all probes specific to that region will have a uniform signal that represents having 2 copies of each chromosome at that position. A CNV is detected by looking for increases (duplication) or decreases (deletion) in signal intensity at individual probes, each of which represent a unique location in the genome. When 25 or more probes targeting contiguous regions of the genome show a reduced signal compared to an individual with no CNV, the test individual can then be said to have a deletion at the location containing the probes that have a reduced signal. Similarly, when 25 or more probes (for example 30 or more probes, or 50 or more probes) targeting contiguous regions of the genome show an increased signal compared to an individual with no CNV, the test individual can then be said to have a duplication at the location containing the probes that have an increased signal. Since the genomic coordinates of each probe are known, CNV size is determined by the coordinates of the probes showing reduced (in the case of a deletion) or increased (in the case of a duplication) signal intensity, and the maximal CNV boundaries are defined by the probes nearest to those showing reduced (deletion) signal or increased (duplication) signal that themselves do not show a reduced (deletion) signal or increased (duplication) signal.

For example, consider an example with oligonucleotide probes each having an arbitrary size of 1 unit for each probe. Probes 1-10 show a normal signal (e.g., as the probe is labeled with a detectable label), probes 11-67 show a reduced signal, and probes 68-1000 show a normal signal again. In this case, there is a deletion that is at least 56 units (67-11=56) in size, and at most 58 units in size (68-10). The CNV boundaries lie somewhere between probes 10 and 11 on the “left” end and between probes 67 and 68 on the “right” end. The same is true for a duplication, but one probes for an increase in signal intensity compared to a subject with no CNV, and duplications must include at least 50 probes to be detectable.

Where non-microarray based hybridization methods are employed to detect the presence or absence of a chromosomal deletion, e.g. a CNV, the size of the CNV can also be determined. For example, in a sequencing embodiment, the number of sequence reads of a particular sequence can be used to make a determination of whether a deletion occurs at the particular chromosomal location. Specifically, the number of sequence reads at a particular genomic DNA location can be compared to the number of sequence reads measured or that would be expected for a sample that does not include the deletion.

As provided above, an oligonucleotide probe or probes designed to hybridize a CNV or portion thereof can be labeled with a detectable label. A “detectable label” is a molecule or material that can produce a detectable (such as visually, electronically or otherwise) signal that indicates the presence and/or concentration of the label in a sample. When conjugated to a nucleic acid such as a DNA probe, the detectable label can be used to locate and/or quantify a target nucleic acid sequence to which the specific probe is directed. Thereby, the presence and/or amount of the target in a sample can be detected by detecting the signal produced by the detectable label. A detectable label can be detected directly or indirectly, and several different detectable labels conjugated to different probes can be used in combination to detect one or more targets.

Examples of detectable labels, which may be detected directly, include fluorescent dyes and radioactive substances and metal particles. In contrast, indirect detection requires the application of one or more additional probes or antibodies, i.e., secondary antibodies, after application of the primary probe or antibody. Thus, in certain embodiments, as would be understood by the skilled artisan, the detection is performed by the detection of the binding of the secondary probe or binding agent to the primary detectable probe. Examples of primary detectable binding agents or probes requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to the oligonucleotides used in the methods of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (TB), tetramethylbenzidine (TMB), 4-chloro-1-naphtol (CN), α-naphtol pyronin (α-NP), o-dianisidine (OD), 5-bromo-4-chloro-3-indolylphosp-hate (BLIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B 1-phosphate/new fuschin (NABP/NF), bromochloroindolyl phosphate/nitroblue tetrazolium (BLIP/NBT), 5-Bromo-4-chloro-3-indolyl-b-d-galactopyrano side (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to any molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents, nucleic acids, or antibodies, etc. Moreover, the skilled artisan would appreciate that each additional binding agent or nucleic acid used to characterize a biological marker of interest (e.g., the CNV genetic markers associated with ASD) may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

In other embodiments, the probes can be indirectly labeled with, e.g., biotin or digoxygenin, or labeled with radioactive isotopes such as 32P and 3H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

Methods for generating arrays are known in the art and include, e.g., photolithographic methods (see, e.g., U.S. Pat. Nos. 5,143,854; 5,510,270; and 5,527,681, each of which is incorporated by reference in its entirety), mechanical methods (e.g., directed-flow methods as described in U.S. Pat. No. 5,384,261), pin-based methods (e.g., as described in U.S. Pat. No. 5,288,514, incorporated by reference in its entirety), and bead-based techniques (e.g., as described in PCT US/93/04145, incorporated by reference in its entirety). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. After hybridization and washing, the array is scanned to determine the position on the array to which the nucleic acid from the sample hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms, e.g., distinct polymorphisms at the same polymorphic site or polymorphisms at different chromosomal sites. Detection blocks may be grouped within a single array or in multiple, separate arrays so that varying conditions (e.g., conditions optimized for particular polymorphisms) may be used during the hybridization.

Additional description of use of oligonucleotide arrays for detection of polymorphisms can be found, for example, in U.S. Pat. Nos. 5,858,659 and 5,837,832, each of which is incorporated by reference in its entirety.

Results of the seizure susceptibility region profiling on a sample from a subject (test sample) may be compared to a biological sample(s) or data derived from a biological sample(s) that is known or suspected to be normal (“reference sample” or “normal sample”). In some embodiments, a reference sample is a sample that is not obtained from an individual having WHS. The reference sample may be assayed at the same time, or at a different time from the test sample.

The results of an assay on the test sample may be compared to the results of the same assay on a reference sample. In some cases, the results of the assay on the reference sample are from a database, or a reference. In some cases, the results of the assay on the reference sample are a known or generally accepted value or range of values by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, deletion presence or absence, deletion size.

In one embodiment, an odds ratio (OR) is calculated for each individual chromosomal deletion measurement. Here, the OR is a measure of association between the presence or absence of an SNP, and an outcome, e.g., seizure susceptibility region deletion positive or negative, or likely to respond to anti-seizure therapy. Odds ratios are most commonly used in case-control studies. For example, see, J. Can. Acad. Child Adolesc. Psychiatry 2010; 19(3): 227-229, which is incorporated by reference in its entirety for all purposes. Odds ratios for each chromosomal deletion can be combined to make an ultimate diagnosis, to select a patient for treatment of seizures, or to predict whether a subject is likely to respond to a particular anti-seizure therapy.

In one embodiment, a specified statistical confidence level may be determined in order to provide a diagnostic confidence level. For example, it may be determined that a confidence level of greater than 90% may be a useful predictor of the presence of a seizure susceptibility region deletion, or to predict whether a subject is likely to respond to therapy for seizures. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and/or the number of genetic markers analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

In one embodiment, a subject is identified as having a deletion of a 197 kbp seizure susceptibility region starting 368 kbp from the terminal end of the short arm of chromosome 4. A subject identified as having a deletion of the seizure susceptibility region is predisposed to seizures. In one embodiment, a subject identified as having a deletion of the seizure susceptibility region comprises a deletion of the entire 197 kbp seizure susceptibility region. In one embodiment, a subject identified as having a deletion of the seizure susceptibility region comprises a deletion of a part of the seizure susceptibility region. In one embodiment, the part of the seizure susceptibility region comprises the PIGG gene. In one embodiment, a subject identified as having a deletion larger than the seizure susceptibility region, wherein the size of the deletion is at least about 500 bp, at least about 1,000 bp, at least about 10,000 bp, at least about 100,000 bp, at least about 1 mega base pairs (Mb), at least about 5 Mb, at least about 10 Mb, at least about 15 Mb, at least about 20 Mb, or at least about 50 Mb.

In one embodiment, a subject identified as having a deletion of a 197 kbp seizure susceptibility region starting 368 kbp from the terminal end of the short arm of chromosome 4 is selected for anti-seizure therapy. In one embodiment, the subject is selected for a particular seizure therapy, e.g., one of the treatments described herein.

Determination of the presence or absence of the deletion of the seizure susceptibility region, and accordingly, selection for treatment of seizures is dependent upon where the at least one CNV occurs in the genome, i.e., whether or not it comprises all or a part of the 197 kbp seizure susceptibility region. Therefore, the CNV location can be mapped to identify a patient for anti-seizure treatment (i.e., selection of the patient for treatment).

As noted above, the 197 kbp seizure susceptibility region encompasses two genes, ZNF721 and PIGG, and one pseudogene, ABCA11P. ZNF721 encodes a zinc-finger-containing protein of unknown function, PIGG encodes a member of the phosphatidylinositol glycan anchor biosynthetic pathway and ABCA11P is a pseudogene with sequence similarity to ATP-binding cassette, subfamily A. Accordingly, in one aspect of the invention, deletion of the seizure susceptibility region may be detected indirectly. For example, a decrease in gene expression as measured by a decrease in mRNA of any one of ZNF721, PIGG, and ABCA11P corresponds to a deletion of the seizure susceptibility region. Similarly, a decrease in the amount of a protein encoded by any one of ZNF721, PIGG, and ABCA11P corresponds to a deletion of the seizure susceptibility region.

In one embodiment, a deletion in the seizure susceptibility region is detected by a decreased amount of PIGG mRNA in comparison to an amount of PIGG mRNA of a control sample. In one embodiment, a deletion in the seizure susceptibility region is detected by a decreased amount of ZNF721 mRNA in comparison to an amount of ZNF721 mRNA of a control sample. In one embodiment, a deletion in the seizure susceptibility region is detected by a decreased amount of ABCA11P mRNA in comparison to an amount of ABCA11P mRNA of a control sample. In one embodiment, a deletion in the seizure susceptibility region is detected by a decreased amount of PIGG mRNA, ZNF721 mRNA, and ABCA11P mRNA in comparison to an amount of PIGG mRNA, ZNF721 mRNA, and ABCA11P mRNA of a control sample. In one embodiment, a decreased amount of mRNA in a test sample in comparison to a control sample indicates a deletion in the seizure susceptibility region.

Detection and quantification of mRNA may be performed using any of a variety of methods available in the art. For example, mRNA may be detected by Northern blot, in situ hybridization (e.g., fluorescent ISH, FISH), and RT-PCR (e.g., real time RT-PCR).

In one embodiment, a deletion in the seizure susceptibility region is detected by a decreased amount of PIGG protein in comparison to an amount of PIGG protein of a control sample. In one embodiment, a deletion in the seizure susceptibility region is detected by a decreased amount of ZNF721 protein in comparison to an amount of ZNF721 protein of a control sample. In one embodiment, a deletion in the seizure susceptibility region is detected by a decreased amount of ABCA11P protein in comparison to an amount of ABCA11P protein of a control sample. In one embodiment, a deletion in the seizure susceptibility region is detected by a decreased amount of PIGG protein, ZNF721 protein, and ABCA11P protein in comparison to an amount of PIGG protein, ZNF721 protein, and ABCA11P protein of a control sample. In one embodiment, a decreased amount of protein in a test sample in comparison to a control sample indicates a deletion in the seizure susceptibility region.

Detection and quantification of protein may be performed using any of a variety of methods available in the art. For example, protein may be detected by western blot, radioimmunoassay (RIA), ELISA, immunohistochemistry, immunoprecipitation, and flow cytometry.

PIGG protein is an enzyme responsible for one step in a biosynthetic pathway that assembles and attaches a phosphatidylinositol glycan (GPI) anchor to over 150 separate proteins in order to direct them to the outer leaflet of the plasma membrane where they carry out various signaling and extracellular functions (Kinoshita, 2014). Deficiencies in GPI anchor synthesis, including those caused by variants in PIGG, underlie congenital disorders of glycosylation, which are associated with infantile encephalopathy, ID, and/or seizures (Makrythanasis et al., 2016). In zebrafish, knock-down of functional members of the GPI biosynthetic pathway results in the failure of the Scn1bb sodium channel to localize to the plasma membrane. Zebrafish scn1bb is the homolog of human SCN1B, mutations in which, or in its human ortholog, SCN1A, are linked etiologically to infantile encephalopathies including Dravet syndrome (Chopra and Isom, 2014). SCN1A and SCN1B proteins may require active PIGG for cell surface expression. Deletion of the seizure susceptibility region, and therefore PIGG, results in a perturbation in the translocation of SCN1A and SCN1B.

In one embodiment, a deletion in the seizure susceptibility region is detected by a decreased amount of SCN1A protein expressed on the cell surface in comparison to an amount of SCN1A protein expressed on the cell surface of a control sample. In one embodiment, a deletion in the seizure susceptibility region is detected by a decreased amount of SCN1B protein expressed on the cell surface in comparison to an amount of SCN1B protein expressed on the cell surface of a control sample. In one embodiment, a deletion in the seizure susceptibility region is detected by a decreased amount of SCN1A and SCN1B proteins expressed on the cell surface in comparison to an amount of SCN1A and SCN1B proteins expressed on the cell surface of a control sample. In one embodiment, the cell is a neural cell. In one embodiment, the cell is an induced pluripotent stem cell (i-PSC). In one embodiment, the neural cell is derived from an iPSC.

Detection and quantification of cell surface protein may be performed using any of a variety of methods available in the art. For example, a protein expressed on a cell surface may be detected by western blot, fluorescence microscopy, immunohistochemistry, and flow cytometry.

### Controlling Seizures

Certain embodiments of the invention provide methods of controlling seizures in a WHS individual. In one embodiment, the WHS subject comprises a deletion of a 197 kbp seizure susceptibility region starting 368 kbp from the terminal end of the short arm of chromosome 4. By “treating” or “controlling” seizures, or seizure activity, as used herein, is meant a reduction in the frequency and/or magnitude of seizures. For example, in one embodiment, the subject has at least one fewer seizures over a time period following treatment in comparison to the number of seizures over an equivalent time period prior to treatment. In one embodiment, the subject has no seizures after treatment. In one embodiment, the subject has 10%, 20%, 30%, 40%, 50%, 60%, 70%, 80%, 90% fewer seizures following treatment. Seizure activity may be monitored, e.g., by questionnaires and/or electroencephalography (EEG). In a preferred embodiment, seizure activity is monitored by EEG.

Various types of seizures include, but are not limited to, tonic-clonic seizures, clonic seizures, tonic seizures (tonic spasms), myoclonic seizures, absence seizures, atonic seizures, complex partial seizures, simple partial seizures, atypical seizures, and status epilepticus. A subject may experience one or more types of seizures. A treatment method may reduce one type of seizure in a subject. A treatment method may reduce more than one type of seizure in a subject. A treatment method may reduce seizures of all types in a subject.

In one embodiment, a method for treating WHS seizures comprises administering an effective amount of an anti-epileptic drug (AED). AEDs include, for example, but are not limited to, Carbamazepine, Chlorazepate, Clobazam, Clonazepam, Diazepam, Dipotassium, Docosahexaenoic acid, Ethosuximide, Felbamate, Fosphenytoin, Gabapentin, Lacosamide, Lamotrigine, Levitiracetam, Levocarnitine, Lorazepam, Midazolam, Oxcarbazepine, Phenobarbital, Phenytoin, Primidone, Propanolol, Rufinamide, Tiagabine, Topiramate, Valproate, Vigabatrin, and Zonisamide. One non-drug treatment of seizures is a ketogenic diet.

Cannabidiol

Cannabinoids, such as tetrahydrocannabivarin (THCV) and cannabidiol (CBD) have been used as anti-convulsants (see, e.g., U.S. Pat. Nos. 9,066,920; 9,125,859; and US 2014/0155456, each of which is herein incorporated by reference in its entirety). One embodiment of the invention provides a method for treating WHS seizures comprising administering an effective amount of CBD to a subject. In one embodiment, the subject comprises a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, the CBD is purified. “Purified” CBD is CBD that exists apart from its native environment (i.e., cannabis) and is therefore not a product of nature. For example, purified CBD is substantially free of other plant material or substantially free of chemical precursors or other chemicals when chemically synthesized. One example of purified CBD is Epidiolex (GW Pharmaceuticals).

In one embodiment, the CBD is a plant extract. In one embodiment, the CBD is present in a Cannabis strain. In one embodiment, the Cannabis strain contains an elevated level of CBD, also referred to as a CBD-rich strain or a high CBD Cannabis. In one embodiment, a CBD-rich strain contains at least 5% CBD by weight. In one embodiment, a CBD-rich strain contains at least 10% CBD by weight. In one embodiment, a CBD-rich strain contains at least 15% CBD by weight. In one embodiment, a CBD-rich strain contains at least 20% CBD by weight. Examples of CBD-rich Cannabis strains containing an increased amount of CBD include, but are not limited to, the cannabis cultivars described in US 2015/0359188, US 2016/0000843, and US 2016/0073566.

In one embodiment, administering CBD reduces the frequency of seizures. In one embodiment, administering CBD eliminates seizures. In one embodiment, CBD is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering CBD eliminates one or more types of seizures the subject experiences. In one embodiment, administering CBD reduces one type of seizure in the subject. In one embodiment, administering CBD reduces more than one type of seizure in the subject. In one embodiment, administering CBD reduces seizures of all types in the subject.

In one embodiment, CBD is administered in combination with one or more AEDs. In one embodiment, CBD is administered in combination with Levetiracetam. In one embodiment, CBD is administered in combination with Valproic acid. In one embodiment, CBD is administered in combination with Levetiracetam and Valproic acid. In one embodiment, CBD is administered in combination with a ketogenic diet. Additional examples of combinations of CBD and one or more AED may be found, e.g., in US 2014/0155456, the disclosure of which is herein incorporated by reference in its entirety.

One embodiment of the invention provides a method for reducing seizure activity comprising administering an effective amount of CBD to a subject identified as having a deletion of a 197 kbp seizure susceptibility region starting 368 kbp from the terminal end of the short arm of chromosome 4. In one embodiment, the deletion was detected by CMA. In one embodiment, the subject has been diagnosed with WHS. In one embodiment, the CBD is purified. In one embodiment, administering CBD reduces the frequency of seizures. In one embodiment, administering CBD eliminates seizures. In one embodiment, CBD is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering CBD eliminates one or more types of seizures the subject experiences. In one embodiment, administering CBD reduces one type of seizure in the subject. In one embodiment, administering CBD reduces more than one type of seizure in the subject. In one embodiment, administering CBD reduces seizures of all types in the subject.

In one embodiment, CBD is administered in combination with one or more AEDs. In one embodiment, CBD is administered in combination with Levetiracetam. In one embodiment, CBD is administered in combination with Valproic acid. In one embodiment, CBD is administered in combination with Levetiracetam and Valproic acid. In one embodiment, CBD is administered in combination with a ketogenic diet.

Vitamin B6

Homozygous deletions and mutations in PIGG give rise to a type of congenital disorder of glycosylation (CDG). Other autosomal recessive conditions caused by the mutation of genes involved in the same phosphatidylinositol glycan anchor biosynthetic pathway are associated with seizures that are amenable to vitamin B6 treatment.

One embodiment of the invention provides a method for treating WHS seizures comprising administering an effective amount of vitamin B6 to a subject. In one embodiment, the subject comprises a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, the vitamin B6 is purified. “Purified” vitamin B6 is vitamin B6 that exists apart from its native environment (e.g., a food source) and is therefore not a product of nature. For example, “purified” vitamin B6 is substantially free of cellular material or culture medium when produced recombinantly, or substantially free of chemical precursors or other chemicals when chemically synthesized.

In one embodiment, administering vitamin B6 reduces the frequency of seizures. In one embodiment, administering vitamin B6 eliminates seizures. In one embodiment, vitamin B6 is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering vitamin B6 eliminates one or more types of seizures the subject experiences. In one embodiment, administering vitamin B6 reduces one type of seizure in the subject. In one embodiment, administering vitamin B6 reduces more than one type of seizure in the subject. In one embodiment, administering vitamin B6 reduces seizures of all types in the subject.

In one embodiment, vitamin B6 is administered in combination with one or more AEDs. In one embodiment, vitamin B6 is administered in combination with Levetiracetam. In one embodiment, vitamin B6 is administered in combination with Valproic acid. In one embodiment, vitamin B6 is administered in combination with Levetiracetam and Valproic acid. In one embodiment, vitamin B6 is administered in combination with a ketogenic diet.

One embodiment of the invention provides a method for reducing seizure activity comprising administering an effective amount of vitamin B6 to a subject identified as having a deletion of a 197 kbp seizure susceptibility region starting 368 kbp from the terminal end of the short arm of chromosome 4. In one embodiment, the deletion was detected by CMA. In one embodiment, the subject has been diagnosed with WHS. In one embodiment, the vitamin B6 is purified.

In one embodiment, administering vitamin B6 reduces the frequency of seizures. In one embodiment, administering vitamin B6 eliminates seizures. In one embodiment, vitamin B6 is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering vitamin B6 eliminates one or more types of seizures the subject experiences. In one embodiment, administering vitamin B6 reduces one type of seizure in the subject. In one embodiment, administering vitamin B6 reduces more than one type of seizure in the subject. In one embodiment, administering vitamin B6 reduces seizures of all types in the subject.

In one embodiment, vitamin B6 is administered in combination with one or more AEDs. In one embodiment, vitamin B6 is administered in combination with Levetiracetam. In one embodiment, vitamin B6 is administered in combination with Valproic acid. In one embodiment, vitamin B6 is administered in combination with Levetiracetam and Valproic acid. In one embodiment, vitamin B6 is administered in combination with a ketogenic diet.

Combination of CBD and Vitamin B6

One embodiment of the invention provides a method for treating WHS seizures comprising administering an effective amount of a combination of vitamin B6 and CBD to a subject. In one embodiment, the subject comprises a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, administering the combination of vitamin B6 and CBD reduces the frequency of seizures. In one embodiment, administering the combination of vitamin B6 and CBD eliminates seizures. In one embodiment, the combination of vitamin B6 and CBD is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering the combination of vitamin B6 and CBD eliminates one or more types of seizures the subject experiences. In one embodiment, administering the combination of vitamin B6 and CBD reduces one type of seizure in the subject. In one embodiment, administering the combination of vitamin B6 and CBD reduces more than one type of seizure in the subject. In one embodiment, administering the combination of vitamin B6 and CBD reduces seizures of all types in the subject.

In one embodiment, the combination of vitamin B6 and CBD is administered in combination with one or more AEDs. In one embodiment, the combination of vitamin B6 and CBD is administered in combination with Levetiracetam. In one embodiment, the combination of vitamin B6 and CBD is administered in combination with Valproic acid. In one embodiment, the combination of vitamin B6 and CBD is administered in combination with Levetiracetam and Valproic acid. In one embodiment, the combination of vitamin B6 and CBD is administered in combination with a ketogenic diet.

One embodiment of the invention provides a method for reducing seizure activity comprising administering an effective amount of a combination of vitamin B6 and CBD to a subject identified as having a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, administering the combination of vitamin B6 and CBD reduces the frequency of seizures. In one embodiment, administering the combination of vitamin B6 and CBD eliminates seizures. In one embodiment, the combination of vitamin B6 and CBD is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering the combination of vitamin B6 and CBD eliminates one or more types of seizures the subject experiences. In one embodiment, administering the combination of vitamin B6 and CBD reduces one type of seizure in the subject. In one embodiment, administering the combination of vitamin B6 and CBD reduces more than one type of seizure in the subject. In one embodiment, administering the combination of vitamin B6 and CBD reduces seizures of all types in the subject.

In one embodiment, the combination of vitamin B6 and CBD is administered in combination with one or more AEDs. In one embodiment, the combination of vitamin B6 and CBD is administered in combination with Levetiracetam. In one embodiment, the combination of vitamin B6 and CBD is administered in combination with Valproic acid. In one embodiment, the combination of vitamin B6 and CBD is administered in combination with Levetiracetam and Valproic acid. In one embodiment, the combination of vitamin B6 and CBD is administered in combination with a ketogenic diet.

Butyrate

As noted above, homozygous deletions and mutations in PIGG give rise to a type of CDG. This subtype of CDG and other autosomal recessive conditions caused by the mutation of genes involved in the same phosphatidylinositol glycan anchor biosynthetic pathway are associated with seizures that are amenable to butyrate treatment.

One embodiment of the invention provides a method for treating WHS seizures comprising administering an effective amount of butyrate to a subject. In one embodiment, the subject comprises a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, the butyrate is purified. In one embodiment, the vitamin B6 is purified. “Purified” butyrate is butyrate that exists apart from its native environment (e.g., a food source) and is therefore not a product of nature. For example, “purified” butyrate is substantially free of cellular material or culture medium when produced recombinantly, or substantially free of chemical precursors or other chemicals when chemically synthesized.

In one embodiment, administering butyrate reduces the frequency of seizures. In one embodiment, administering butyrate eliminates seizures. In one embodiment, butyrate is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering butyrate eliminates one or more types of seizures the subject experiences. In one embodiment, administering butyrate reduces one type of seizure in the subject. In one embodiment, administering butyrate reduces more than one type of seizure in the subject. In one embodiment, administering butyrate reduces seizures of all types in the subject.

In one embodiment, butyrate is administered in combination with one or more AEDs. In one embodiment, butyrate is administered in combination with Levetiracetam. In one embodiment, butyrate is administered in combination with Valproic acid. In one embodiment, butyrate is administered in combination with Levetiracetam and Valproic acid. In one embodiment, butyrate is administered in combination with a ketogenic diet.

One embodiment of the invention provides a method for reducing seizure activity comprising administering an effective amount of butyrate to a subject identified as having a deletion of a 197 kbp seizure susceptibility region starting 368 kbp from the terminal end of the short arm of chromosome 4. In one embodiment, the deletion was detected by CMA. In one embodiment, the subject has been diagnosed with WHS. In one embodiment, the butyrate is purified.

In one embodiment, administering butyrate reduces the frequency of seizures. In one embodiment, administering butyrate eliminates seizures. In one embodiment, butyrate is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering butyrate eliminates one or more types of seizures the subject experiences. In one embodiment, administering butyrate reduces one type of seizure in the subject. In one embodiment, administering butyrate reduces more than one type of seizure in the subject. In one embodiment, administering butyrate reduces seizures of all types in the subject.

In one embodiment, butyrate is administered in combination with one or more AEDs. In one embodiment, butyrate is administered in combination with Levetiracetam. In one embodiment, butyrate is administered in combination with Valproic acid. In one embodiment, butyrate is administered in combination with Levetiracetam and Valproic acid. In one embodiment, butyrate is administered in combination with a ketogenic diet.

Combination of Vitamin B6 and Butyrate

One embodiment of the invention provides a method for treating WHS seizures comprising administering an effective amount of a combination of vitamin B6 and butyrate to a subject. In one embodiment, the subject comprises a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, administering the combination of vitamin B6 and butyrate reduces the frequency of seizures. In one embodiment, administering the combination of vitamin B6 and butyrate eliminates seizures. In one embodiment, the combination of vitamin B6 and butyrate is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering the combination of vitamin B6 and butyrate eliminates one or more types of seizures the subject experiences. In one embodiment, administering the combination of vitamin B6 and butyrate reduces one type of seizure in the subject. In one embodiment, administering the combination of vitamin B6 and butyrate reduces more than one type of seizure in the subject. In one embodiment, administering the combination of vitamin B6 and butyrate reduces seizures of all types in the subject.

In one embodiment, the combination of vitamin B6 and butyrate is administered in combination with one or more AEDs. In one embodiment, the combination of vitamin B6 and butyrate is administered in combination with Levetiracetam. In one embodiment, the combination of vitamin B6 and butyrate is administered in combination with Valproic acid. In one embodiment, the combination of vitamin B6 and butyrate is administered in combination with Levetiracetam and Valproic acid. In one embodiment, the combination of vitamin B6 and butyrate is administered in combination with a ketogenic diet.

One embodiment of the invention provides a method for reducing seizure activity comprising administering an effective amount of a combination of vitamin B6 and butyrate to a subject identified as having a deletion of a 197 kbp seizure susceptibility region starting 368 kbp from the terminal end of the short arm of chromosome 4. In one embodiment, the deletion was detected by CMA. In one embodiment, the subject has been diagnosed with WHS.

In one embodiment, administering the combination of vitamin B6 and butyrate reduces the frequency of seizures. In one embodiment, administering the combination of vitamin B6 and butyrate eliminates seizures. In one embodiment, the combination of vitamin B6 and butyrate is administered to treat one or more of tonic-clonic seizures, clonic seizures, tonic spasms, myoclonic seizures, absence seizures, atonic seizures, complex partial seizures, simple partial seizures, atypical seizures, and status epilepticus. In one embodiment, administering the combination of vitamin B6 and butyrate eliminates one or more types of seizures the subject experiences. In one embodiment, administering the combination of vitamin B6 and butyrate reduces one type of seizure in the subject. In one embodiment, administering the combination of vitamin B6 and butyrate reduces more than one type of seizure in the subject. In one embodiment, administering the combination of vitamin B6 and butyrate reduces seizures of all types in the subject.

In one embodiment, the combination of vitamin B6 and butyrate is administered in combination with one or more AEDs. In one embodiment, the combination of vitamin B6 and butyrate is administered in combination with Levetiracetam. In one embodiment, the combination of vitamin B6 and butyrate is administered in combination with Valproic acid. In one embodiment, the combination of vitamin B6 and butyrate is administered in combination with Levetiracetam and Valproic acid. In one embodiment, the combination of vitamin B6 and butyrate is administered in combination with a ketogenic diet.

### EXAMPLES

The present invention is further illustrated by reference to the following Examples. However, it should be noted that these Examples, like the embodiments described above, are illustrative and are not to be construed as restricting the scope of the invention in any way. The references cited in the Examples are incorporated by reference in their entireties for all purposes.

### Example 1

### Identification of a 4P Terminal Region Associated with Seizures in Wolf-Hirschhorn Syndrome

Wolf-Hirschhorn syndrome (WHS) is a contiguous gene deletion syndrome involving variable size deletions of the 4p16.3 region. Seizures are frequently, but not always, associated with WHS. In order to determine if the size and location of the deleted region correlates with seizure presentation, chromosomal microarray analysis (CMA) was used to finely map the breakpoints of copy number variants (CNVs) in 48 individuals with WHS. Seizure phenotype data were collected through parent-reported answers to a comprehensive questionnaire and supplemented with available medical records.

Because WHS is a contiguous gene deletion syndrome, loss of one copy of a single gene or the synergistic effects of loss of two or more genes could give rise to the features of WHS. One such gene, LETM1, falls within WHSCR2 and has been proposed as a candidate seizure gene (Endele et al. Genomics 1999; 60:218-25; Jiang et al. Science 2009; 326:144-7; Schlickum et al. Genomics 2004; 83:254-61; Zhang et al. Cereb Cortex 2014; 24:2533-40), due to the suggested pathogenic link between mitochondrial dysfunction and epilepsy (Zsurka et al. Lancet Neurol 2015; 14:956-66). The protein encoded by LETM1 localizes to mitochondria and functions in Ca2+ homeostasis, oxidative stress prevention and ATP generation (Doonan et al. FASEB J 2014; 28:4936-49; Hart et al. Dis Model Mech 2014; 7:535-45; Jiang et al. Proc Natl Acad Sci USA 2013; 110:E2249-54). Consistent with the hypothesis that LETM1 is a seizure susceptibility gene, heterozygous Letm1± mice, as well as rats with a lentiviral-mediated Letm1 knockdown, demonstrate increased seizure susceptibility in response to kainic acid or pilocarpine seizure induction (Zhang et al. Cereb Cortex 2014; 24:2533-40; Jiang et al. Proc Natl Acad Sci USA 2013; 110:E2249-54).

Despite this evidence, LETM1 is not likely to be the sole seizure susceptibility gene in the 4p region. In recent years, increased awareness of the diagnostic features of WHS within the medical community, coupled with the advent of high-resolution cytogenetic methods, has led to the identification and characterization of submicroscopic 4p deletions. Some of these deletions suggest that LETM1 deletion is neither necessary nor sufficient for the expression of a seizure phenotype in individuals with WHS (South et al. Eur J Hum Genet 2008; 16:45-52; Luo et al. Hum Mol Genet 2011; 20:3769-78; Andersen et al. Eur J Hum Genet 2014; 22:464-70; Bayindir et al. Eur J Med Genet 2013; 56:551-5; Van Buggenhout et al. J Med Genet 2004; 41:691-8; Engbers et al. Eur J Hum Genet 2009; 17:129-32; Faravelli et al. Am J Med Genet A 2007; 143A:1169-73; Izumi et al. Am J Med Genet A 2010; 152A:1028-32; Misceo et al. Gene 2012; 507:85-91) and have led to the proposal of alternative candidate seizure genes (Zollino et al. Epilepsia 2014; 55:849-57).

Using high-density microarray analysis combined with parent-reported seizure phenotypes, this study was performed in order to identify a seizure-susceptibility region. A relatively large, 48-individual cohort was recruited through partnership with the 4p-Support Group (Vanzo et al. Am J Med Genet A 2014; 164A:1619-21). Evaluation of deletion coordinates and seizure phenotypes in this cohort identified a likely seizure susceptibility region within the 751 kbp terminal region of chromosome 4p. Combining these data with cases described in the literature, this seizure susceptibility region was narrowed to a region 197 kbp in size that includes two genes and one pseudogene. Also described are the types of seizures associated with WHS observed in the cohort and the response to antiepileptic medications reported by the cohort. This study demonstrates the potential value of using high-resolution CMA for the diagnosis and medical management of seizures associated with WHS.

### Patient Cohort

Forty-eight individuals with a diagnosis of WHS, along with their parents, consented to this study during one of two national meetings of the 4p-Support Group held in July 2012 in Indianapolis, Ind., and July 2014 in Harrisburg, Pa. (Vanzo et al. Am J Med Genet A 2014; 164A:1619-21). In total, 28 females and 20 males with WHS, with an average age of 11.2 years, were recruited into this study (Table 2).

TABLE 2
Clinical and molecular cytogenetic findings of the study cohort

| Total participants | 48 | | Female:male | 28:20 | |
|---|---|---|---|---|---|
| Average age | 11.2 | years | Range | 0.9-38 | years |
| Initial | 88% | (30/34) | Initial | 12% | (4/34) |
| diagnosis by | | | diagnosis by | | |
| karyotype/FISH | | | CMA | | |

| | | | | |
|---|---|---|---|---|
| Size range of | 1.7-33.9 | Mbp | Number of | 28-207 |
| 4p deletion | | | genes deleted | |
| Individuals | 29% | (14/48) | Average | 3.2 Mbp |
| with a | | | size of | (range 51.3 kbp |
| second CNV | | | second CNV | to 8.3 Mbp) |

| | | |
|---|---|---|
| Individuals | Interstitial: 5 | Terminal: 29 |
| with only a | | |
| 4p deletion by | | |
| deletion type | | |

### Clinical and Molecular Cytogenetic Studies

All cytogenetic analyses were performed through regular clinical services in clinical laboratory improvement amendments (CLIA)-certified laboratories. All genomic coordinates for CNVs are reported herein using human reference sequence hg19/GRCh37. All patients (exceptions noted below) were physician referred for clinical microarray testing to Lineagen (Salt Lake City, Utah, USA). Testing for these patients was done using Lineagen's custom 2.8M probe SNP-based microarray described in WO 2014/055915, the disclosure of which is incorporated herein by reference in its entirety. The Affymetrix Chromosome Analysis Suite (ChAS) software was used for CNV detection (Affymetrix, Santa Clara, Calif., USA). Exceptions to the above were as follows: a 2.7M probe Cytogenetics Array (Affymetrix) was performed by Lineagen on patients 35 and 40. Patients 12, 17 and 45 obtained prior clinical CMA from other CLIA laboratories, and these patients provided a copy of their laboratory reports for analysis.

### Phenotype Analysis

Phenotype data were collected through parent-reported answers to a comprehensive questionnaire developed by Battaglia et al. (Am J Med Genet C Semin Med Genet 2008; 148C:246-51) (see online supplementary materials). This questionnaire captures the health, medical profile, developmental history, and treatment responses of individuals with WHS. For the present study, the focus was on the presence or absence of seizures, age of seizure onset, types of seizures, antiepileptic drugs (AEDs) used and responses to these AEDs, as well as responses to the ketogenic diet. For cases with incomplete, contradictory or unclear parental responses, medical records of patients were consulted. When available medical records were also incomplete, ‘no answer’ is indicated in the relevant text and tables.

### Statistical Methods

Two-tailed Fisher's exact test was used for comparing the group of individuals with interstitial 4p deletions to the group with terminal deletions and their seizure phenotypes. Significance was defined as p<0.01.

### Results

Table 2 shows the age and gender characteristics of this study cohort. Prior to this study, the initial diagnosis of WHS was made by individuals' physicians using clinical assessment and a combination of G-banded karyotyping and FISH, or CMA (Table 2). Fourteen individuals did not indicate which method(s) were used in their initial diagnosis.

Physician-ordered CMA was performed on the 44 individuals comprising the cohort who had not already had chromosomal microarray testing done as part of their diagnostic work up. The array used was a custom 2,784,985-probe chromosomal microarray to achieve high-resolution mapping of the 4p deletion breakpoints, as well as to define the breakpoints of any other clinically reportable CNVs that could be detected (Table 3).

TABLE 3
CNV breakpoints, gender, age at time of analysis, age of seizure onset, and AEDs for individuals
with 4p deletions and no other clinically reportable CNVs (henceforth designated “individuals
with only 4p deletions.”). All coordinates are given in hg19/GRCh37.

| | | Deletion | | | Seizures: | |
|---|---|---|---|---|---|---|
| | CNV coordinates | size | | Age | age of onset | Seizure medications |
| Patient | (hg19) | (Mbp) | Gender | (years) | (months) | (AEDs) |
| 1 | chr4: 68345-32587789 | 32.5 | F | 3.8 | 5 | Phenobarbital |
| 2 | chr4: 68345-22799761 | 22.7 | F | 14.8 | 11 | For SE: Diazepam. |
| | | | | | | Carbemazepine, |
| | | | | | | Topiramate |
| 3 | chr4: 68345-19797868 | 19.7 | M | 7.2 | 5 | Diazepam and Lorazepam |
| | | | | | | for SE. Levitiracetam, |
| | | | | | | Topiramate, and |
| | | | | | | Clonazepam in combination. |
| | | | | | | Levitiracetam only. |
| 4 | chr4: 68345-19258986 | 19.2 | M | 16.5 | 6 | Chlorazepate Dipotassium |
| | | | | | | and Lacosamide and |
| | | | | | | “many others.” |
| 5 | chr4: 68345-18958105 | 18.9 | F | 2.8 | 16 | For SE: Levitiracetam, |
| | | | | | | Diazepam, rectal Diazepam. |
| | | | | | | For seizure control: |
| | | | | | | Levitiracetam, rectal Diazepam |
| 6 | chr4: 68345-16452492 | 16.4 | F | 38.0 | 5 | Phenobarbital, Valproate. |
| | | | | | | Weaned off all seizure |
| | | | | | | meds at age 21 |
| 7 | chr4: 68345-15891049 | 15.8 | F | 8.0 | 17 | Phenobarbital, Diazepam. |
| | | | | | | Has been seizure free |
| | | | | | | for past two years. |
| | | | | | | Ketogenic diet. |
| 8 | chr4: 68345-15338783 | 15.3 | M | 4.0 | No | Lamotrigine |
| | | | | | answer¹ | |
| 9 | chr4: 68345-15067905 | 15.0 | F | 26.5 | 14 | Phenobarbital, |
| | | | | | | Valproic acid. |
| 10 | chr4: 1025119-15582327 | 14.6 | M | 3.7 | 18 | Advised not to medicate |
| | | | | | | due to lack of seizures. |
| 11 | chr4: 68345-13578589 | 13.5 | F | 20.5 | 8 | Diazepam for SE. |
| | | | | | | Phenobarbital, |
| | | | | | | Phenobarbital/Valproate, |
| | | | | | | Valproate/Clonazepam, |
| | | | | | | Valproate/Levitiracetam, |
| | | | | | | Levitiracetam only |
| | | | | | | since July 2006. |
| 12 | chr4: 49450-11487322 | 11.4 | F | 1.0 | No | Levitiracetam, |
| | | | | | answer¹ | Valproate, |
| | | | | | | Levocarnitine |
| 13 | chr4: 68345-10621914 | 10.6 | F | 7.5 | 9 | Topiramate, Levitiracetam, |
| | | | | | | Lamotrigine. Phenobarbital |
| 14 | chr4: 68345-10255806 | 10.2 | F | 5.0 | 6 | Diazepam, Midazolam, |
| | | | | | | Propanolol for SE. |
| | | | | | | Phenobarbital, |
| | | | | | | Clonazepam, Oxcarbazepine, |
| | | | | | | Clonazepam, Ethosuximide, |
| | | | | | | Gabapentin, Phenytoin, |
| | | | | | | Valproic acid, Felbamate, |
| | | | | | | Topiramate, Zonisamide, |
| | | | | | | Lacosamide, Levitiracetam. |
| | | | | | | Clobazam Lorazepam. |
| 15 | chr4: 68345-9785068 | 9.7 | F | 9.0 | 16 | For SE: Lorazepam, Diazepam, |
| | | | | | | Phenytoin. For other seizures: |
| | | | | | | Carbemazepine, Levitiracetam. |
| | | | | | | Seizure free for three years |
| 16 | chr4: 68345-7670607 | 7.6 | M | 6.0 | No | No answer |
| | | | | | answer¹ | |
| 17 | chr4: 965069-7686694 | 6.7 | M | 1.5 | n/a² | n/a |
| 18 | chr4: 68345-6335151 | 6.3 | F | 2.3 | 14 | Levitiracetam, Diazepam |
| 19 | chr4: 68345-6146360 | 6.1 | F | 3.5 | 9 | Diazepam, Docosahexaenoic acid |
| 20 | chr4: 68345-5595216 | 5.5 | F | 22.0 | No | Phenobarbital, Valproate |
| | | | | | answer¹ | from 24 mo to 7 yr |
| 21 | chr4: 1701018-7102682 | 5.4 | F | 10.0 | n/a² | n/a |
| 22 | chr4: 68345-5418070 | 5.3 | F | 18.0 | 11 | Carbemazepine |
| 23 | chr4: 113981-5087478 | 5.0 | M | 11.0 | 12 | Diazepam in case |
| | | | | | | of emergency |
| | | | | | | but never used |
| 24 | chr4: 1682255-6055232 | 4.4 | M | 8.0 | n/a² | n/a |
| 25 | chr4: 68345-4426571 | 4.4 | M | 15.0 | 24 | No answer |
| 26 | chr4: 68345-4288168 | 4.2 | M | 20.0 | 24 | No answer |
| 27 | chr4: 68345-4214933 | 4.1 | M | 5.0 | 42 | Levetiracetam |
| 28 | chr4: 68345-3956051 | 3.9 | F | 5.0 | 10 | Diazepam |
| 29 | chr4: 68345-2283825 | 2.2 | F | 6.7 | No | Levitiracetam, Topiramate, |
| | | | | | answer¹ | Lamotrigine, Lorazepam, |
| | | | | | | Clonazepam, Phenytoin, |
| | | | | | | Diazepam |
| 30 | chr4: 68345-2115175 | 2.0 | F | 9.0 | No | Levitiracetam, Lamotrigine |
| | | | | | answer¹ | Phenobarbital |
| 31 | chr4: 68345-2110649 | 2.0 | M | 11.3 | No | Valproate |
| | | | | | answer¹ | |
| 32 | chr4: 68345-2009432 | 1.9 | F | 15.0 | No | Topiramate, Levitiracetam, |
| | | | | | answer¹ | Clobazam, Ketogenic diet |
| 33 | chr4: 68345-1740152 | 1.7 | F | 6.0 | No | Topiramate, Oxcarbazepine, |
| | | | | | answer¹ | Lamotrigine, Levitiracetam, |
| | | | | | | Valproic Acid, Ketogenic |
| | | | | | | diet, Diazepam |
| 34 | chr4: 750979-2009432 | 1.3 | F | 11.0 | n/a² | n/a |
| | | Average | | | Average age of onset | |
| | | size: 9.6 | | | (months): 13.4 +/− 8.7 | |

¹Age of onset was not provided; patient is known to have seizures.
²Patient does not have seizures; therefore age of onset is not applicable (n/a).
SE, status epilepticus.

Twenty-nine percent of the cohort had a second deletion or duplication involving either chromosome 4 or another chromosome. This percentage is in keeping with previous studies of chromosomal rearrangements associated with WHS4 (Table 2).

Some of the second CNVs in the cohort are pathogenic, while others are of unknown clinical significance. The pathogenic CNVs are associated with developmental delay, ID, autism spectrum disorder, dysmorphic features and seizures. The breakpoints of all patients' 4p deletions, as well as the breakpoints of the second CNV if present, and the association of this second CNV to any clinical features are listed in Tables 3 and 4.

TABLE 4
CNV breakpoints, gender, age at time of study, and age of seizure onset for individuals with a
4p deletion and a second clinically reportable CNV. All coordinates are given in hg19/GRCh37.

| | 4p | 4p | Additional | Additional | | | | Seizures: | |
|---|---|---|---|---|---|---|---|---|---|
| | CNV | deletion | CNV | CNV | Pathogenicity | | | age of | Seizure |
| | coordinates | size | coordinates | size | of second | | Age | onset | medications |
| Patient | (hg19) | (Mbp) | (hg19)¹ | (Mbp) | CNV | Sex | (years) | (months²) | (AEDs) |
| 35 | chr4: 68345- | 33.9 | chr19: 56455446- | 0.58 | VOUS | F | 0.9 | 6 | Levitiracetam |
| | 33934217 | | 57033092 (dup) | | | | | | |
| 36 | chr4: 68345- | 23.0 | chr2: 110873834- | 0.11 | VOUS; Joubert | F | 27.0 | 16 years | For SE: rectal |
| | 23113681 | | 110980107 (del) | | carrier due | | | old, | Diazepam. |
| | | | | | to NPHP1 | | | initially | Remains on |
| | | | | | deletion | | | associated | Valproate |
| | | | | | | | | with | but has not |
| | | | | | | | | menarche | had seizure |
| | | | | | | | | | for several |
| | | | | | | | | | years |
| 37 | chr4: 68345- | 22.9 | chr16: 21931247- | 0.51 | Pathogenic: 16p12.2 | M | 4.5 | | Levitiracetam, |
| | 22970801 | | 22442007 (del) | | microdeletion syndrome. | | | | Topiramate |
| | | | | | This syndrome is | | | | |
| | | | | | associated with high | | | | |
| | | | | | penetrance and | | | | |
| | | | | | variable expressivity | | | | |
| | | | | | and includes the | | | | |
| | | | | | following: DD, ID, | | | | |
| | | | | | autism, microcephaly, | | | | |
| | | | | | congenital heart | | | | |
| | | | | | defects, hypotonia, | | | | |
| | | | | | seizures. [Girirajan | | | | |
| | | | | | et al. Nat Genet 2010; | | | | |
| | | | | | 42: 203-9; Cooper et al. | | | | |
| | | | | | Nat Genet 2011; 43: 838- | | | | |
| | | | | | 46; Shaikh et al. Genome | | | | |
| | | | | | Res 2009; 19: 1682-90; | | | | |
| | | | | | Girirajan and Eichler. | | | | |
| | | | | | Hum Mol Genet 2010; | | | | |
| | | | | | 19: R176-87; Itsara | | | | |
| | | | | | et al. Am J Hum Genet | | | | |
| | | | | | 2009; 84: 148-61] | | | | |
| 38 | chr4: 68345- | 21.9 | chr1: 246840624- | 2.4 | VOUS: case reports | M | 23.0 | 9 | ATCH |
| | 21981129 | | 249224684 (dup) | | associate 1q44 trisomy | | | | injections for |
| | | | | | with ID, speech delay, | | | | infantile spasms, |
| | | | | | facial features, heart | | | | Diazepam for SE. |
| | | | | | defects, macrocephaly, | | | | Clonazepam, |
| | | | | | ASD, seizures[Lenzini | | | | Topiramate, |
| | | | | | et al. Genet Test Mol | | | | Lamotrigine, |
| | | | | | Biomark 2009; 13: 79- | | | | Diazepam, |
| | | | | | 86; Villa et al. J Med | | | | Phenytoin, |
| | | | | | Genet 2000; 37: 612-5]AM | | | | Phenobarbital, |
| | | | | | | | | | Levitiracetam, |
| | | | | | | | | | Tiagabine, |
| | | | | | | | | | Gabapentin, |
| | | | | | | | | | Carbamazepine |
| 39 | chr4: 5762133- | 21.0 | chr4: 5488819- | 0.27 | VOUS: This is a | M | 25.0 | 18 | Valproate |
| | 26720441 | | 5760770 (dup) | | duplication adjacent | | | | |
| | | | | | and distal to the | | | | |
| | | | | | deletion in 4p16.1. | | | | |
| | | | | | NB: Due to the CMA | | | | |
| | | | | | results, this individual's | | | | |
| | | | | | diagnosis was changed | | | | |
| | | | | | from WHS to proximal | | | | |
| | | | | | 4p deletion syndrome. | | | | |
| 40 | chr4: 68345- | 17.9 | chr4: 17983558- | 0.58 | VOUS, likely benign | M | 6.7 | 6 | Lorazepam |
| | 17983528 | | 18562949 (dup) | | | | | | for SE. |
| | | | | | | | | | Valproate, |
| | | | | | | | | | Rufinamide, |
| | | | | | | | | | Clonazepam, |
| | | | | | | | | | Levitiracetam, |
| | | | | | | | | | Phenobarbital, |
| | | | | | | | | | Primidone, |
| | | | | | | | | | Topiramate, |
| | | | | | | | | | Lorazepam, |
| | | | | | | | | | Diazepam, |
| | | | | | | | | | Clobazam, |
| | | | | | | | | | Vigabatrin, |
| | | | | | | | | | Zonisamide, |
| | | | | | | | | | Ketogenic diet. |
| 41 | chr4: 68345- | 15.2 | chr4: 15310408- | 1.9 | VOUS | F | 14.0 | 8 | Topiramate and |
| | 15305951 | | 17226923 (dup) | | | | | | Clonazepam |
| | | | | | | | | | for SE. |
| | | | | | | | | | Phenobarbital, |
| | | | | | | | | | Clonazepam, |
| | | | | | | | | | Carbamazepine, |
| | | | | | | | | | Topiramate |
| 42 | chr4: 68345- | 9.4 | chr8: 158048- | 7 | VOUS | M | 20.0 | 7 | Diazepam, and |
| | 9501651 | | 7112571 (dup) | | | | | | Lorazepam |
| | | | | | | | | | for SE. |
| | | | | | | | | | Phenytoin, |
| | | | | | | | | | Phenobarbital, |
| | | | | | | | | | Valproate, |
| | | | | | | | | | Carbamazepine. |
| | | | | | | | | | Has been off |
| | | | | | | | | | seizure meds |
| | | | | | | | | | since age 15 |
| 43 | chr4: 68345- | 7.4 | chr8: 146082484- | 0.21 | VOUS | M | 12.0 | 9 | Diazepam, |
| | 7442049 | | 146295771 (dup) | | | | | | Fosphenytoin |
| | | | | | | | | | for SE. |
| | | | | | | | | | Phenytoin, |
| | | | | | | | | | Topiramate, |
| | | | | | | | | | Clonazepam, |
| | | | | | | | | | Lamotrigine, |
| | | | | | | | | | Carbamazepine, |
| | | | | | | | | | Phenobarbital, |
| | | | | | | | | | Valproate, |
| | | | | | | | | | Zonisamide, |
| | | | | | | | | | Oxcarbazepine |
| 44 | chr4: 68345- | 6.2 | chr3: 124342797- | 0.051 | VOUS; Deletion includes | M | 5.0 | 12 | Valproic |
| | 6257188 | | 124394169 (del) | | the KALRN gene[Russell | | | | acid |
| | | | | | et al. Nat Commun | | | | |
| | | | | | 2014; 5: 4858]AM | | | | |
| 45 | chr4: 68345- | 4.1 | chr8: 158048- | 6.8 | VOUS | M | 2.0 | 16 | Valproic |
| | 4165335 | | 6999114 (dup) | | | | | | acid |
| 46 | chr4: 68345- | 3.9 | chr12: 173786- | 8.2 | Pathogenic: 12p trisomy | F | 3.0 | No answer | Diazepam, |
| | 3941740 | | 8393815 (dup) | | syndrome. 12p trisomy | | | | Clobazam, |
| | | | | | syndrome is characterized | | | | Vigabatrin |
| | | | | | by seizures, DD, and | | | | |
| | | | | | hypotonia, unique facial | | | | |
| | | | | | features, and hearing | | | | |
| | | | | | loss[Benussi et al. | | | | |
| | | | | | Genet Test Mol Biomark | | | | |
| | | | | | 2009; 13: 199-204]AM | | | | |
| 47 | chr4: 68345- | 3.9 | chr12: 173786- | 8.2 | Pathogenic: 12p trisomy | F | 19.5 | 8 | Diazepam, |
| | 3927887 | | 8393815 (dup) | | syndrome. 12p trisomy | | | | Phenobarbital, |
| | | | | | syndrome is characterized | | | | Phenytoin, |
| | | | | | by seizures, DD, and | | | | Levitiracetam, |
| | | | | | hypotonia, unique facial | | | | Valproic |
| | | | | | features, and hearing | | | | acid with |
| | | | | | loss[Benussi et al. | | | | Phenytoin, |
| | | | | | Genet Test Mol Biomark | | | | Topiramate |
| | | | | | 2009; 13: 199-204]AM | | | | |
| 48 | chr4: 68345- | 2.4 | chr22: 42932261- | 8.3 | Pathogenic: Phelan- | F | 22.0 | 15 | Lorazepam |
| | 2437290 | | 51197838 (dup) | | McDermid Syndrome. DD, | | | | for SE, |
| | | | | | hypotonia, ASD, and absent | | | | Levitiracetam |
| | | | | | or delayed speech | | | | |
| | | Average | | Average | | | | Average | |
| | | size of 4p | | size of | | | | age of | |
| | | deletion | | additional | | | | onset | |
| | | (Mbp): 13.8 | | CNV | | | | (months), | |
| | | | | (Mbp): 3.2 | | | | excluding | |
| | | | | | | | | Patient 36 | |
| | | | | | | | | as an | |
| | | | | | | | | outlier: | |
| | | | | | | | | 10.4 +/− 4.2 | |

¹For additional CNVs, deletion (del) or duplication (dup) of the region is indicated in parentheses following the CNV coordinates.
²Age of seizure onset of Patient 36 is given in years.
Abbreviations: VOUS, variant of unknown significance, ABN, abnormal CNV, ID, intellectual disability, DD, developmental delay, ASD, autism spectrum disorder

Consistent with previous studies (Battaglia et al. Brain Dev 2005; 27:362-4; Battaglia et al. Pediatrics 1999; 103:830-6; Battaglia et al. Dev Med Child Neurol 2009; 51:373-80; Battaglia et al. Epilepsia 2003; 44:1183-90), it was found that 90% (43/48) of the cohort had seizures, which were of early onset (Tables 3 and 4), were often brought on by fever (25/41 individuals reported having febrile seizures) and tended to wane in frequency during the preteen years. All seizure types surveyed (tonic-clonic, tonic, clonic, myoclonic, absence, atonic, complex partial, simple partial, atypical and status epilepticus) were detected in this cohort. The seizure types most commonly reported in the WHS cohort are shown in Table 5.

TABLE 5
Most frequently reported seizure types

| | | Individuals with |
|---|---|---|
| | Individuals with | 4p deletion and an |
| Type | only 4p deletion | additional CNV |
| Tonic-clonic | 19/24 (79%) | 9/13 (69%) |
| Absence | 12/24 (50%) | 8/13 (62%) |
| Status epilepticus | 10/24 (42%) | 7/13 (54%) |
| Complex partial | 8/24 (33%) | 3/13 (23%) |
| Myoclonic | 5/24 (21%) | 5/13 (38%) |

### Mapping a Seizure Susceptibility Candidate Region

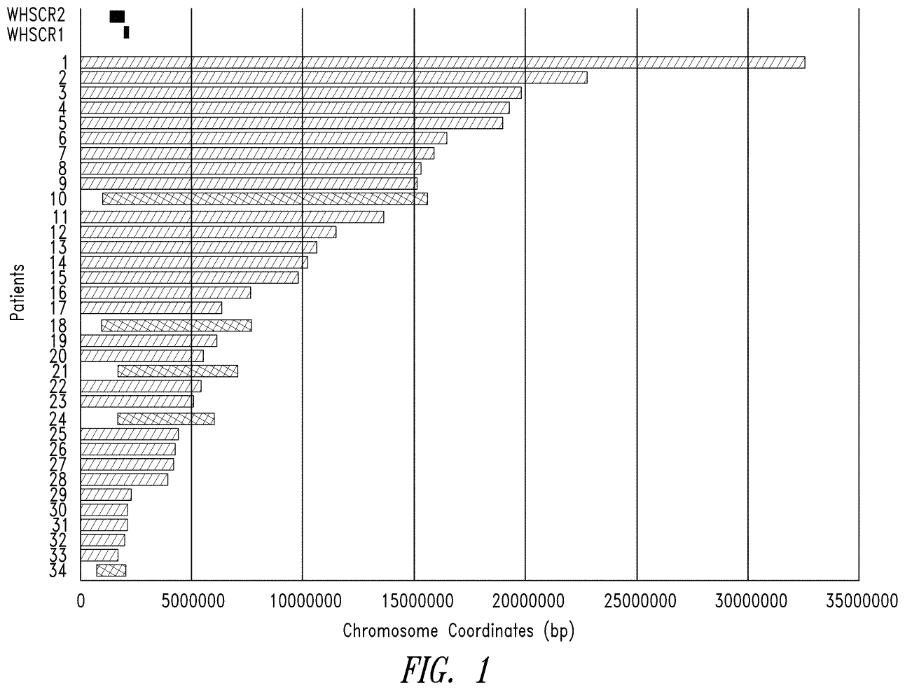

To identify a region conferring a genetic susceptibility to seizures, the 34 patients in the cohort with only 4p deletions were evaluated. FIG. 1 shows the deletions of this group aligned by size and location. All individuals in this group have deletions that encompass both critical regions WHSCR1 and WHSCR2 except for patient 33, whose deletion only overlaps WHSCR2 but not WHSCR1.

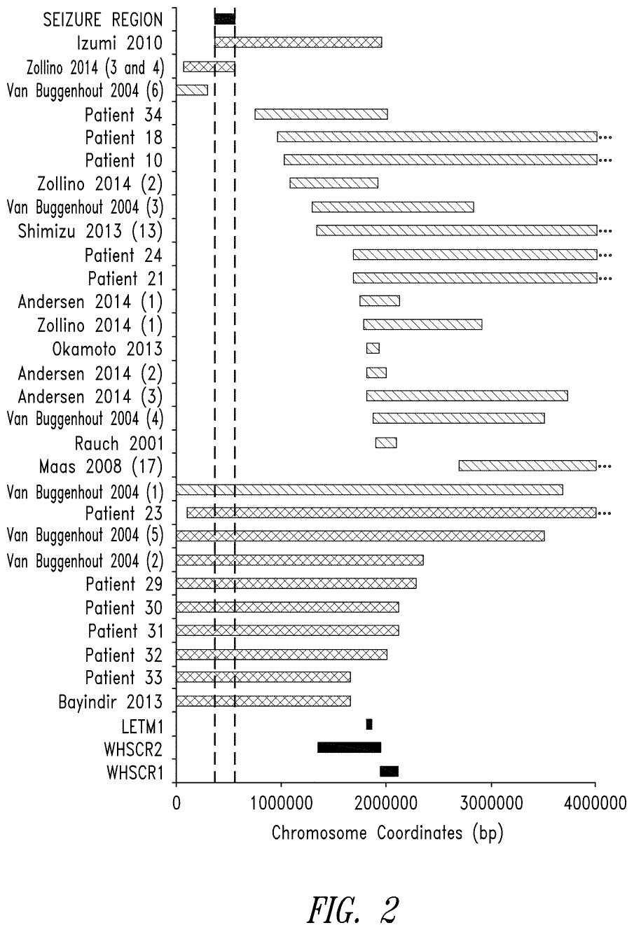

It was asked whether 4p deletion size and genetic content correlate with seizure severity by first examining the records of the five individuals with the smallest terminal deletions in the cohort, patients 29-33 (FIGS. 1 and 2). Their deletions range in size from 1.7 to 2.2 Mbp. Typically, individuals with small 4p terminal deletions less than 3.5-6 Mbp in size exhibit the mildest phenotypes, including seizure phenotypes (Maas et al. J Med Genet 2008; 45:71-80; Shimizu et al. Am J Med Genet A 2014; 164A:597-609; Zollino et al. Am J Med Genet 2000; 94:254-61; Zollino et al. Am J Med Genet C Semin Med Genet 2008; 148C:257-69). Notably, four of these five individuals (patients 29, 31, 32 and 33) reported having severe seizure phenotypes, indistinguishable in terms of seizure types, frequency or response to AEDs (Table 3) from the rest of the cohort with larger deletion sizes. Patient 33 is noteworthy because her deletion does not remove LETM1, the purported candidate seizure gene, yet her seizures are consistent with WHS. Thus, it was observed that in the cohort, small terminal 4p deletions including one that does not include LETM1 can result in severe seizure phenotypes.

In contrast, four individuals, patients 18, 21, 24 and 34, were identified who did not have seizures as well as one additional individual, patient 10, who is considered as not having seizures, as explained below. All of these individuals have interstitial deletions that leave, minimally, the terminal 751 kbp of chromosome 4p intact (FIG. 1). Patient 10 had the largest interstitial 4p deletion, 14.6 Mb in size, who had one febrile seizure at age 1.5 years associated with a kidney infection. Having an isolated febrile seizure is an unusual presentation for WHS-associated epilepsy; in accordance with his medical records and parent answers on our survey, we scored him as not having WHS-related seizures. Taken together, these data show that deletion of the terminal 751 kbp of chromosome 4p, not monosomy of LETM1, correlates with an epileptic phenotype (p=3.59e-6) using a two-tailed Fisher's exact test (Table 6).

TABLE 6
Calculation of Two-tailed Fisher's Exact test

| | 751 kbp terminal | 751 kbp terminal |
|---|---|---|
| | region intact | region deleted |
| Seizures: No | 5 | 0 |
| Seizures: Yes | 0 | 29 |

We turned to the literature to determine if other rare interstitial deletions or small terminal deletions would support or refute the hypothesis that the deletion of the terminal region of 4p correlates with a seizure phenotype. Nine additional cases of non-related individuals with WHS and without seizures have been previously described in the literature (Maas et al. J Med Genet 2008; 45:71-80; Van Buggenhout et al. J Med Genet 2004; 41:691-8; Zollino et al. Epilepsia 2014; 55:849-57; Shimizu et al. Am J Med Genet A 2014; 164A:597-609; Okamoto et al. Am J Med Genet A 2013; 161A:1465-9; Rauch et al. Am J Med Genet 2001; 99:338-42). Their reported deletion sizes and locations are shown in FIG. 2, along with the deletions of patients from the study cohort who lack seizures. Also included in FIG. 2 are three small interstitial deletions described by Andersen et al. (Eur J Hum Genet 2014; 22:464-70), all of which encompass at least portions of the WHSC1 and LETM1 genes. The three individuals with these deletions show features of WHS but do not meet the minimal diagnostic criteria for the syndrome and do not have seizures (Andersen et al. Eur J Hum Genet 2014; 22:464-70). Strikingly, 16 out of 17 individuals without seizures have interstitial deletions, most of which result in monosomy of LETM1 while leaving the terminal 751 kbp intact. The exception to this observed correlation was an 11-year-old girl without seizures who had a ˜3.7 Mbp terminal deletion that also removes LETM1 (Van Buggenhout 2004, patient 1) (FIG. 2).

The corresponding chromosome coordinates for all these patients are given in Table 7. “SEIZURE REGION” is the candidate 197 kbp seizure susceptibility region, the SRO between patients described in Izumi et al (2010) and Zollino et al 2014). WHS Critical regions are labeled “WHSCR” and “WHSCR2,” respectively. Patient identifiers are in parentheses and correspond to the number given to them in their respective papers (cited). For example, “Zollino 2014 (3 and 4)” is the label for deletion shared by siblings, patients 3 and 4 in Zollino et al., Epilepsia 2014; 55:849-57. All coordinates are given in hg19/GRCh37. Some deletion sizes from older reports had to be inferred because the mapping of breakpoints was done using FISH probes and not by microarray analysis.

TABLE 7
Coordinates for all deletions helping to
define a seizure susceptibility region

| | Coordinates | Seizures? |
|---|---|---|
| SEIZURE REGION | chr4: 367691-564593 | |
| Izumi 2010 [Am J Med | chr4: 367691-1948108 | Yes |
| Genet A, 152A: 1028-32] | | |
| Zollino 2014 (3 and 4) | chr4: 71552-564593 | Yes |
| [Epilepsia, 55: 849-57] | | |
| Van Buggenhout 2004 (6) | chr4: 0-300000 | No |
| [J Med Genet, 41: 691-8] | | |
| 34 | chr4: 750979-2009432 | No |
| 18 | chr4: 965069-7686694 | No |
| 10 | chr4: 1025119-15582327 | No |
| Zollino 2014 (2) | chr4: 1079721-1919855 | No |
| [Epilepsia, 55: 849-57] | | |
| Van Buggenhout 2004 | chr4: 1283478-2833478 | No |
| (3) [J Med Genet, 41: 691-8] | | |
| Shimizu 2013 (13) [Am J | chr4: 1339023-10645858 | No |
| Med Genet A, 164A: 597-609] | | |
| 24 | chr4: 1682255-6055232 | No |
| 21 | chr4: 1701018-7102682 | No |
| Andersen 2014 (1) [Eur J | chr4: 1743630-2120247 | No |
| Hum Genet EJHG, 22: 464-70] | | |
| Zollino 2014 (1) | chr4: 1778765-2909499 | No |
| [Epilepsia, 55: 849-57] | | |
| Okamoto 2013 [Am J Med | chr4: 1822203-1931042 | No |
| Genet A, 161A: 1465-9] | | |
| Andersen 2014 (2) [Eur J | chr4: 1827029-1997169 | No |
| Hum Genet EJHG, 22: 464-70] | | |
| Andersen 2014 (3) [Eur J | chr4: 1828867-3724595 | No |
| Hum Genet EJHG, 22: 464-70] | | |
| Van Buggenhout 2004 | chr4: 1880226-3505324 | No |
| (4) [J Med Genet, 41: 691-8] | | |
| Rauch 2001 [Am J Med | chr4: 1906575-2093987 | No |
| Genet, 99: 338-42] | | |
| Maas 2008 (17) [J Med | chr4: 2700000-14800000 | No |
| Genet, 45: 71-80] | | |
| Van Buggenhout 2004 | chr4: 0-3679582 | No |
| (1) [J Med Genet, 41: 691-8] | | |
| Van Buggenhout 2004 | chr4: 0-3505324 | Yes |
| (5) [J Med Genet, 41: 691-8] | | |
| Van Buggenhout 2004 | chr4: 0-2351677 | Yes |
| (2) [J Med Genet, 41: 691-8] | | |
| Bayindir 2013 [Eur J Med | chr4: 68345-1729442 | Yes |
| Genet, 56: 551-5] | | |
| 33 | chr4: 68345-1740152 | Yes |
| 23 | chr4: 113981-5087478 | Yes |
| LETM1 | chr4: 1813206-1857974 | |
| WHSCR2 | chr4: 1345236-1945236 | |
| WHSCR1 | chr4: 1945236-2110236 | |

One individual described by Van Buggenhout et al. (J Med Genet 2004; 41:691-8) was a clinically normal patient with a history of multiple miscarriages and no seizures. This patient was found to have a 0.3 Mbp terminal deletion (Van Buggenhout 2004 patient 6, FIG. 2) using a BAC array. While the lower resolution of BAC arrays must be taken into account, the deletion in this individual nevertheless suggests that a deletion encompassing approximately 0.3 Mbp of the 4p terminus does not contribute to the seizure phenotype or any other characteristic traits of WHS.

Next, the literature was searched for examples of individuals with seizures who had the smallest described terminal and interstitial deletions of chromosome 4p. The deletions of 12 such individuals, including five from the study cohort, are shown in FIG. 2 (red bars). Eight individuals in this group have terminal deletions and four have interstitial deletions, all of which affect at least the distal-most 500 kbp of chromosome 4p. Most notably, Zollino et al. (Epilepsia 2014; 55:849-57) have recently described two siblings, with a paternally inherited 564 kbp terminal deletion (FIG. 2, Zollino 2014, patients 3 and 4). Both siblings, as well as their father, have a history of seizures.

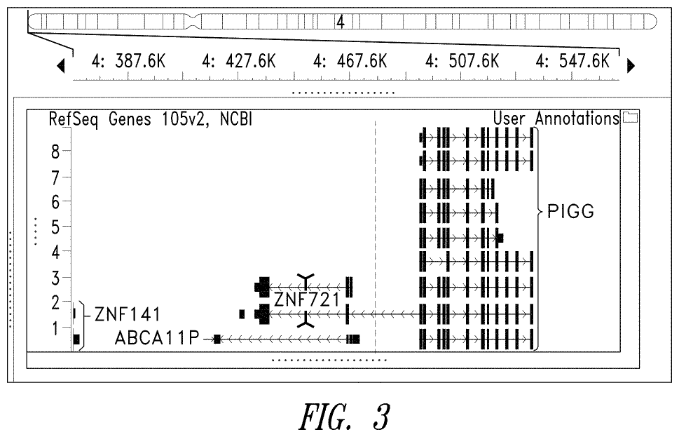

A 1.58 Mbp interstitial deletion of a 33-month-old girl overlaps with the deletions of patients 3 and 4 from Zollino et al. (Epilepsia 2014; 55:849-57). This patient, described by Izumi et al. (Am J Med Genet A 2010; 152A:1028-32), presented with a typical WHS seizure phenotype. The SRO shared by the deletions of these three patients can therefore be used to define a seizure susceptibility region 197 kbp in length, starting with the distal coordinate defined by the Izumi patient and the proximal coordinate defined by the two Zollino siblings (FIGS. 2 and 3). There are two genes and one pseudogene in this region: ZNF721, encoding a zinc-finger containing protein of unknown function, PIGG, a member of the phosphatidylinositol glycan anchor biosynthetic pathway, and ABCA11P, a pseudogene with sequence similarity to ATP-binding cassette, subfamily A genes (FIG. 3).

As the study cohort and cases described in the literature have shown, individuals with interstitial 4p deletions that leave this candidate region intact (with the exception of patient 1 from Van Buggenhout et al. J Med Genet 2004; 41:691-8) do not have seizures. Conversely, deletion of this region gives rise to seizures. These observations suggest that deletion of this region is both necessary and sufficient for the seizure phenotype in individuals with WHS.

### Treatment Responses

Study participants reported 19 different AEDs, as well as the ketogenic diet and homeopathic approaches, to control seizures, with varying degrees of success (Tables 3, 4 and 8). The responses of the four most commonly used seizure medications in this cohort are shown in Table 8, with levetiracetam and valproic acid showing the most positive responses within this group. These observations are consistent with previous studies reporting that valproic acid, used alone or in combination with ethosuximide, is the effective treatment for atypical absences common to individuals with WHS (Battaglia et al. Dev Med Child Neurol 2009; 51:373-80; Battaglia et al. Am J Med Genet C Semin Med Genet 2008; 148C:241-3).

TABLE 8
Responses to the four most commonly reported seizure medications

| | | | | Valproic |
|---|---|---|---|---|
| | Phenobarbital | Levetiracetam | Topiramate | acid |
| | (n = 13) | (n = 13) | (n = 11) | (n = 11) |
| Negative | 5 | 1 | 4 | 2 |
| reports | | | | |
| Positive | 0 | 4 | 1 | 2 |
| reports | | | | |

The reported responses are summarized in Table 8. AEDs were scored as positive if the patient's parents reported without prompting that the drug gave a significant and observable increase in control over seizures. AED responses were scored as negative if the patients' parents reported a negative reaction (allergic reaction or other) without prompting that caused them to stop using that drug, or if the drug conferred no control over seizures.

In summary, because seizures affect approximately 90% of all individuals with WHS and can greatly influence the quality of life for these individuals, the analysis was focused on seizures. By fine mapping the 4p deletion breakpoints of the study cohort, a 751 kbp terminal 4p candidate seizure region was identified. The deletion of this region correlated strongly with the presence of seizures, and its preservation, as in cases of the interstitial WHS deletions described above, correlated with the absence of seizures. Rare interstitial and submicroscopic terminal deletions described in the literature not only support the idea that deletion of this region is necessary for seizure phenotype but also support the idea that its deletion is sufficient for predisposition to seizures. In particular, three individuals described in the literature, two of whom are siblings, allowed the boundaries of the candidate seizure susceptibility region to be further refined to a locus 197 kbp in size, starting 368 kbp from the terminal end of chromosome 4.

This study demonstrated that the use of whole genome CMA for the genetic characterization of individuals with WHS is valuable, since it provides a significantly higher resolution of breakpoint coordinates than does karyotyping. Additional CNVs frequently occur in this population (South et al. Eur J Hum Genet 2008; 16:45-52), yet on average are smaller than would be detectable even by high-resolution karyotyping, and can therefore be easily missed. In addition, the presence or absence of the terminal 197 kbp deletion is most effectively detected using CMA.

### Example 2

### The Effect of PIGG Insufficiency on Seizures

The identification of the relatively small candidate seizure region now affords the opportunity to create loss-of-function knockouts of candidate genes in model organisms to confirm that haploinsufficiency of such genes is sufficient to increase seizure susceptibility and also to perform functional studies that will further elucidate the mechanism of these genes' functions in health and disease. Using such an approach, precision medicine for complex genetic disorders such as contiguous gene disorders becomes possible.

The 197 kbp seizure susceptibility region described in the previous Example encompasses two genes and one pseudogene. ZNF721 encodes a zinc-finger-containing protein of unknown function, PIGG encodes a member of the phosphatidylinositol glycan anchor biosynthetic pathway and ABCA11P is a pseudogene with sequence similarity to ATP-binding cassette, subfamily A. While not much is known about the biological function of ZNF721, several intriguing lines of evidence indicate PIGG as an excellent candidate seizure susceptibility gene.

PIGG encodes one of 26 members of a biosynthetic pathway involved in assembling and attaching the phosphatidylinositol glycan (GPI) anchor to a group of over 150 proteins (Kinoshita T. Proc Jpn Acad Ser B Phys Biol Sci 2014; 90:130-43). The GPI anchor serves to attach these proteins to the outer leaflet of the plasma membrane where they carry out various signaling and extracellular functions. Deficiencies in GPI anchor synthesis have been linked to disorders of congenital glycosylation, all of which are autosomal recessive and are associated with infantile encephalopathy, ID, and/or seizures (Kinoshita T. Proc Jpn Acad Ser B Phys Biol Sci 2014; 90:130-43; Chiyonobu et al. J Med Genet 2014; 51:203-7; Ilkovski et al. Hum Mol Genet 2015; 24:6146-59). Further work is necessary to characterize PIGG's role as a candidate seizure susceptibility gene.

If its deletion alone is sufficient to cause seizures, it would be the first description of haploinsufficiency for a GPI anchor biosynthetic gene. This may be consistent with the proposed importance of stoichiometry in the PIGG protein's role in the biosynthetic pathway, in which it functions as a catalytic component and competes with phosphatidylinositol glycan anchor biosynthesis protein, class O (PIGO) for binding to phosphatidylinositol glycan anchor biosynthesis protein, class F (PIGF) in order to add an ethanolamine-phosphate side chain to a mannose moiety (Kinoshita T. Proc Jpn Acad Ser B Phys Biol Sci 2014; 90:130-43). Alternatively, deletion of one copy of PIGG always occurs in the context of the deletion of other 4p terminal genes in cases of WHS; it may be that the deletion of a combination of genes in the WHS region acts synergistically to predispose individuals to seizures.

In order to determine if deletion of PIGG is sufficient to cause seizure, a mouse model may be used. For example, PIGG^{+/+}, PIGG^{+/-}, and PIGG^{-/-} mice can be generated to examine whether deletion of one and/or two copies of PIGG results in a seizure phenotype.

### Example 3

### Treating WHS Seizures with Cannabidiol

The availability of chromosomal microarray analysis (CMA) has led to evolution of our understanding of the genomic variation within WHS and its correlation with phenotype (Maas et al., 2008; Battaglia et al., 2015; South et al., 2008). Recently, we described a novel candidate region for the seizures associated with WHS (Ho et al., 2016). The most plausible candidate gene in the region, PIGG, encodes an enzyme responsible for one step in a biosynthetic pathway that assembles and attaches a phosphatidylinositol glycan (GPI) anchor to over 150 separate proteins in order to direct them to the outer leaflet of the plasma membrane where they carry out various signaling and extracellular functions (Kinoshita, 2014). Deficiencies in GPI anchor synthesis, including those caused by variants in PIGG, underlie congenital disorders of glycosylation, which are associated with infantile encephalopathy, ID, and/or seizures (Makrythanasis et al., 2016).

The identification of PIGG as potential critical contributor to the seizures in WHS has opened the door to potential therapeutic strategies in these patients by virtue of our observation that the GPI pathway constitutes a possible mechanistic link with the complex seizures characteristic of another genetic condition, Dravet Syndrome (Chopra and Isom, 2014; Battaglia and Carey, 2005). Nakano et al., (2010) demonstrated in zebrafish that lack or knock-down of functional members of the GPI biosynthetic pathway results in the failure of the Scn1bb sodium channel to localize to the plasma membrane. Zebrafish scn1bb is the homolog of human SCN1B, mutations in which, or in its human ortholog, SCN1A, are linked etiologically to infantile encephalopathies including Dravet syndrome (Chopra and Isom, 2014).

Beyond this potential mechanistic link, there are significant similarities shared between WHS and Dravet syndrome. Seizures in both have a complex pattern, can be prolonged, are often brought on by febrile episodes, and are often intractable to pharmacotherapies, leading to cognitive, motor and behavioral impairment (Chopra and Isom, 2014; Battaglia and Carey, 2005). Dravet syndrome is characterized by early-onset seizures including febrile, afebrile, generalized/unilateral clonic, myoclonic, focal, and atypical absence seizures. These seizures can be prolonged and often are intractable to pharmacotherapies, leading to cognitive, motor and behavioral impairment (Chopra et al. Epilepsy Curr Am Epilepsy Soc 2014; 14:86-9). Individuals with WHS display a distinctive electroclinical pattern resembling the severe myoclonic epilepsy of infancy or Dravet syndrome (Battaglia et al. Brain Dev 2005; 27:362-4). In addition, some patients with a milder presentation of WHS-related dysmorphologies are sometimes first suspected of having Dravet syndrome, as attested by published studies in which SCN1A sequencing was conducted and found to be negative in at least two cases (Bayindir et al. Eur J Med Genet 2013; 56:551-5; Zollino et al. Epilepsia 2014; 55:849-57) until the true cause, a deletion of the 4p terminus, was identified. The EEG pattern in WHS is distinctively similar to that observed in Dravet syndrome (Battaglia and Carey, 2005). Furthermore, carbamazepine and lamotrigine have been shown to exacerbate seizures in both individuals with WHS as well as individuals with Dravet syndrome (Battaglia et al. GeneReviews. Seattle, Wash.: University of Washington, Seattle, 1993.2015:1-18; Brunklaus et al. Brain J Neurol 2012; 135:2329-36).

In zebrafish, there is an ortholog of SCN1A that corresponds to human SCN1B that has also been linked to Dravet syndrome, designated scn1bb. The Rohon-Beard neurons of zebrafish require functional Scn1bb protein, as well as the phosphatidylinositol biosynthetic pathway, for touch sensitivity. Nakano et al. (Development 2010; 137:1689-98) showed that zebrafish mutants that lack functional members of the phosphatidylinositol biosynthetic pathway, or morpholino knockdown of members of this pathway, result in the failure of the sodium channel Scn1bb to localize correctly to the plasma membrane. This observation could provide an intriguing mechanistic link between seizures in WHS and Dravet syndrome (Chopra et al. Epilepsy Curr Am Epilepsy Soc 2014; 14:86-9).

A recent Phase III trial using a relatively pure preparation of cannabidiol (CBD) demonstrated control of seizures in individuals with Dravet syndrome with seizures that were previously uncontrolled by at least four anti-epileptic prescription drugs. Earlier this year, GW Pharma announced positive results of a two arm pivotal Phase 3 study of its investigational cannabidiol (CBD) medication, Epidiolex® in 120 refractory patients with Dravet syndrome, with a median reduction in monthly seizure episodes of 39 percent compared to only 13 percent on placebo (p=0.01) over the 14-week treatment period compared with the 4-week baseline observation period (GWPharma—GW Pharmaceuticals Announces Positive Phase 3 Pivotal Study Results for Epidiolex® (cannabidiol)). The median baseline convulsive seizure frequency per month was 13. This drug has both Orphan Drug Designation and Fast Track Designation from the U.S. Food and Drug Administration (FDA) in the treatment of Dravet syndrome, and more recently comparable results in the clinically similar condition, Lennox-Gastaut syndrome.

Based upon this marked clinical similarity and the potential mechanistic link, Markham et al. specifically inquired about use of cannabinoids in any form in an online survey of parents from the 4p-Support Group concerning the response of their child with WHS to specific seizure treatments. Roughly 5% (5/95) indicated use of such alternative agents, however it is likely that this represents under-reporting as the survey was not anonymous and such agents are not legal in many jurisdictions still today. Of those who reported use of cannabinoids, 80% noted a reduction in seizure frequency of over 50%, as well as related benefits in terms of reduced side effects from lowered AED therapy dosage (Markham, L. et al., 2016).

Detyniecki and Hirsch (2016) opined in their editorial on a recent, apparently positive open-label interventional trial with CBD (Devinsky et al., 2016), that there is potential for a large placebo effect with highly motivated parents in such anecdotal reports and open-label uncontrolled studies. Filloux (2015) has commented thoughtfully on the need for “real science” in approaching the issue of cannabinoid use in epilepsy in general. While acknowledging these challenges, the noted mechanistic links and clinical similarities between WHS and Dravet syndrome, and the sound studies supporting CBD use in the latter condition, indicate that serious consideration should be given to CBD use in WHS individuals who are not achieving good seizure control with minimized AED side effects and optimal quality of life.

In order to determine if WHS individuals may respond favorably to CBD for seizure control, patients having a deletion of the 197 kbp seizure susceptibility region are selected for the study. Patients having the seizure susceptibility region deletion may be detected using, e.g., chromosomal microarray. Seizure activity can be monitored by parental answers to questionnaires and/or EEG. Analysis of EEG readings of patients before and after administration of CBD is preferred. If seizure activity is reduced (e.g., decreased number and/or frequency of seizures) following administration of CBD, then CBD controls seizure activity.

### Example 4

### Treating WHS Seizures with Vitamin B6 and Butyrate

Individuals with homozygous or compound heterozygous mutations in PIGG have a seizure-related condition. There are other known autosomal recessive and X-linked conditions associated with mutations in genes that function in the same way as PIGG, all of which are associated with seizures. The conditions have all been classified as a subtype of Congenital Disorders of Glycosylation (CDG). Vitamin B6 (Murakami et al., Nihon Rinsho. 2015; 73(7):1227-37) and butyrate (Almeida et al., Biochim Biophys Acta. 2009; 1792(9):874-80) have been shown to be efficacious in treating seizures of this subtype of CDG.

Therefore, in order to determine if WHS individuals benefit from vitamin B6 and butyrate as a treatment for seizures, patients having a deletion of the 197 kbp seizure susceptibility region are selected for the study. Patients having the seizure susceptibility region deletion may be detected using, e.g., chromosomal microarray. Seizure activity can be monitored by parental answers to questionnaires and/or EEG. Analysis of EEG readings of patients before and after administration of vitamin B6 and butyrate is preferred. If seizure activity is reduced (e.g., decreased number and/or frequency of seizures) following administration of vitamin B6 alone, butyrate alone, and/or a combination of vitamin B6 and butyrate, then vitamin B6, butyrate, and/or the combination of vitamin B6 and butyrate controls seizure activity, respectively.

### Example 5

### Effectiveness of Seizure Treatments in WHS

In a study of epilepsy in WHS involving 87 cases, the early childhood onset and seizure types were described. Seizures usually occur within the first three years of life, and are initially unilateral clonic or tonic, or generalized tonic-clonic (Battaglia et al 2009). Nearly 50% of individuals also develop absence seizures later on in childhood. While seizure control is achievable using anti-epileptic medications, a portion of this population develop life-threatening status epilepticus seizures and some never achieve control. While it has been reported that most individuals with WHS respond well to valproic acid, formal study of valproic acid has not been done. A recent report also reported a good response to levetiracetam in a single patient (Karalok et al., Childs Nery Syst. 2016 January; 32(1):9-11). These studies also suggested that epilepsy in WHS can be well controlled if patients are started with the right seizure treatment.

The idea that specific seizure treatments work better for individuals with a particular genetic condition is not new. In fact, multiple studies have been conducted to determine the effectiveness of various seizure treatments in other genetic causes of epilepsy. One study demonstrated that bromide and valproic acid were most effective for individuals with Dravet syndrome (Shi et al., Brain & Development. 2015 Jul. 13). They also found evidence that carbamazepine has no benefit in 78% of Dravet patients, and aggravates seizures in 20% of Dravet patients. Thus, carbamazepine is contraindicated in the Dravet population. In contrast, a retrospective survey was conducted among patients with 15q Duplication syndrome and found that patients generally responded well to valproic acid, levetiracetam, and carbamazepine (Conant et al., Epilepsia. 2014 March; 55(3):396-402).

To date, there are no published studies evaluating seizure treatment in the WHS population involving a large cohort. Accordingly, this study assessed current seizure treatments for WHS in a relatively large cohort, determined whether certain treatments, if any, are more successful than others in this population, and whether any treatments are detrimental to seizure control.

### Methods

Cohort

This study and all recruitment materials were approved by the University of Utah IRB (IRB_00064655). The cohort for this study was recruited from the 4p-Support Group, which is comprised of parents and caretakers of individuals with WHS. Inclusion criteria were: 1) a genetic diagnosis of WHS and 2) at least one seizure treatment had been used for seizure control. An invitation to participate in the survey was sent out to all members of the 4p-Support Group email list (representing approximately 300 individuals with WHS) and posted on the 4p-Support Group's Facebook page. Individuals self-identifying as a parent or caretaker of an individual with WHS and a history of seizures, who had tried at least one seizure treatment, were invited to participate. The invitation described the purpose of the study and provided a link to the online survey.

Survey Instrument

The survey was developed by expanding the seizure history and treatment sections of a previous survey used by Ho et al (J Med Genet 2016; 53:256-263). The online survey was hosted by the HIPAA-compliant platform, RedCAP, and consisted of four sections. The first section collected general demographic information about the individual with WHS. Two additional questions were added to confirm whether the individual had genetic testing to confirm the diagnosis of WHS and ascertain which type of genetic testing had been completed. The second section focused on seizure history. This included age of onset, types of seizures, seizure duration, status epilepticus (seizures lasting longer than 30 minutes), and whether the individual still experiences seizures or is seizure free. The third section asked about antiepileptic drugs (AEDs) used for seizure treatment. Participants were asked to list each AED or combination of AEDs tried in chronological order. For each AED or combination of AEDs participants were asked about duration, effect on number of seizures, side effects, and reasons for discontinuing that AED or combination of AEDs. There were also questions about whether the individual was on a ketogenic diet or using cannabis oil during that time. Space for additional comments about each AED or combination of AEDs tried was also provided. The fourth section contained questions about use of ketogenic diet or cannabis oil while in the absence of AEDs. Again participants were asked about duration, effect on number of seizures, side effects, and reasons for discontinuing. In the case of cannabis oil use, participants were asked to specify whether pure cannabis oil or a THC mixture was used.

Prior to launching the survey, cognitive interviews were conducted with four mothers to individuals with WHS. These interviews were used to assess clarity and understandability of questions. Cognitive interviews were conducted by phone with each mother individually. The survey was available online for four weeks in March 2016.

Statistical Methods

The levels of seizure control outcome were grouped into five categories: increased number of seizure, no change, less than 50% reduction, >50% reduction, and complete control.

When a medication was used multiple times (or when looking at combined medication), the seizure control outcome was scored as the best response among all of them. The most frequently used 5 medications reported in this cohort are phenobarbital, diazepam, lamotrigine, valproic acid (sodium valproate) and levetiracetam. We used frequency and percentages to describe the seizure control outcome of these five medications. The Kruskal-Wallis test was performed to evaluate the level of seizure control achieved with these five medications. The Wilcoxon rank sum test was performed to evaluate the hypothesis of the different seizure control outcomes between carbamazepine and levetiracetam. Comparison of the seizure control outcome based on whether levetiracetam was used first or later, was performed by the Cochran Armitage test. Statistical analysis was conducted using SAS 9.4 (SAS Inc., N.C.).

### Results

Demographics

A total of 164 surveys were returned. Answers from 66 respondents were excluded from analysis because the surveys were incomplete. Analyses were performed on the remaining 98 completed surveys. Invitations to participate were sent to families representing approximately 300 individuals with WHS. Thus the estimated response rate is 33%.

Table 9 summarizes the characteristics of the cohort: which parent completed the survey on the individual's behalf, the age and gender of individuals with WHS, and the method used to determine the genetic diagnosis of WHS.

TABLE 9
Participant Demographics and Seizure History

| | N (%) |
|---|---|
| Completing Survey | |
| Mothers | 89 (91%) |
| Fathers | 9 (9%) |
| Gender of WHS Individuals | |
| Females | 61 (62%) |
| Males | 37 (38%) |
| Age of WHS Individuals | Age |
| Range | 4 months-37 years |
| Mean | 9.5 years |
| Median | 7 years |
| Genetic Testing | N (%) |
| CMA | 59 (60%) |
| Karyotype | 52 (53%) |
| FISH | 49 (50%) |

Seizures

Age of seizure onset ranged from 1 day to 4 years of age. The mean age of onset was 11 months and the median was 9 months. Types of seizures experienced included tonic-clonic, absence, tonic, myoclonic, focal motor, atonic, subclinical, and infantile spasms. Of note, all individuals who experienced infantile spasms also experienced all other seizure types listed. When asked which seizure type occurs most frequently, the three top results were tonic-clonic (33%), absence (27%), and myoclonic (19%). Fifty-nine (60%) individuals experienced at least one episode of status epilepticus, defined as a seizure lasting 30 minutes or longer. Seizure control was felt to be achieved in 77 (79%) of individuals.

Antiepileptic Drug (AED) Treatments

The number of combinations of AEDs tried ranged from 1-14, with a mean of 3.26 and median of 3. Individuals who tried only one AED made up 33% of the cohort. While 23 different AEDs were represented in this data set, statistical analysis was only performed on carbamazepine and the 5 most commonly used AEDs (diazepam, lamotrigine, levetiracetam, phenobarbital, valproic acid).

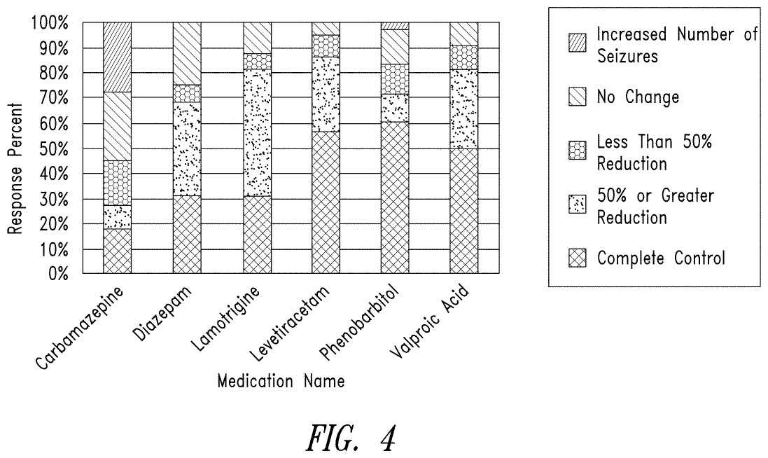

Seizure control outcome was broken down into five categories: complete seizure control, 50% or greater reduction, less than 50% reduction, no change, and increased number of seizures (see Table 10). Comparison of seizure control outcome for diazepam, lamotrigine, levetiracetam, phenobarbital, and valproic acid was done using the Kruskal-Wallis Test (see FIG. 4). The p-value was insignificant at 0.2105, indicating that the five most commonly used drugs had the same effectiveness in controlling seizures. For most patients, this means greater than or equal to a 50% seizure reduction.

TABLE 10
Frequency table for seizure control outcome for 5 medications

| | | | Percent | |
|---|---|---|---|---|
| Medications | Seizure control outcome | N | (%) | P value |
| Phenobarbital | Increased number of seizure | 1 | 2.78 | 0.2105 |
| (n = 36) | No change | 5 | 13.89 | |
| | Less than 50% reduction | 4 | 11.11 | |
| | ≥50% reduction | 4 | 11.11 | |
| | Complete control | 22 | 61.11 | |
| Diazepam | No change | 4 | 25 | |
| (n = 16) | Less than 50% reduction | 1 | 6.25 | |
| | ≥50% reduction | 6 | 37.5 | |
| | Complete control | 5 | 31.25 | |
| Lamotrigine | No change | 2 | 12.5 | |
| (n = 16) | Less than 50% reduction | 1 | 6.25 | |
| | ≥50% reduction | 8 | 50 | |
| | Complete control | 5 | 31.25 | |
| Valproic Acid or | No change | 3 | 9.38 | |
| Sodium valproate | Less than 50% reduction | 3 | 9.38 | |
| (n = 32) | ≥50% reduction | 10 | 31.25 | |
| | Complete control | 16 | 50 | |
| Levetiracetam | No change | 3 | 5.17 | |
| (n = 58) | Less than 50% reduction | 5 | 8.62 | |
| | ≥50% reduction | 17 | 29.31 | |
| | Complete control | 33 | 56.9 | |

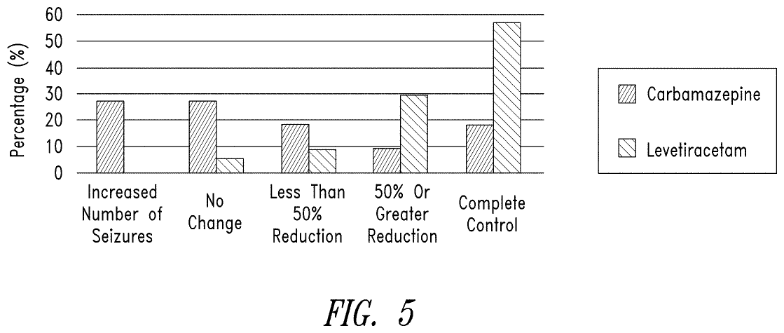

Since carbamazepine has been indicated to have poor outcomes in patients with WHS in the literature (Battaglia et al., 2015. Am J Med Genet Part C Semin Med Genet. 169C:216-223), seizure control outcome using carbamazepine was compared to outcome for levetiracetam using the Wilcoxon Rank sum test (see Table 11 and FIG. 5). Levetiracetam was chosen for this comparison because it represented the largest number of individuals. The p-value was significant at 0.0398.

TABLE 11
Frequency table of seizure control outcome
for Carbamazepine and Levetiracetam

| | | | Percent | |
|---|---|---|---|---|
| Medications | Seizure control outcome | N | (%) | P value |
| Carbamazepine | Increased number of seizure | 3 | 27.27 | 0.0398 |
| (n = 11) | No change | 3 | 27.27 | |
| | Less than 50% reduction | 2 | 18.18 | |
| | ≥50% reduction | 1 | 9.09 | |
| | Complete control | 2 | 18.18 | |
| Levetiracetam | No change | 3 | 5.17 | |
| (n = 58) | Less than 50% reduction | 5 | 8.62 | |
| | ≥50% reduction | 17 | 29.31 | |
| | Complete control | 33 | 56.9 | |

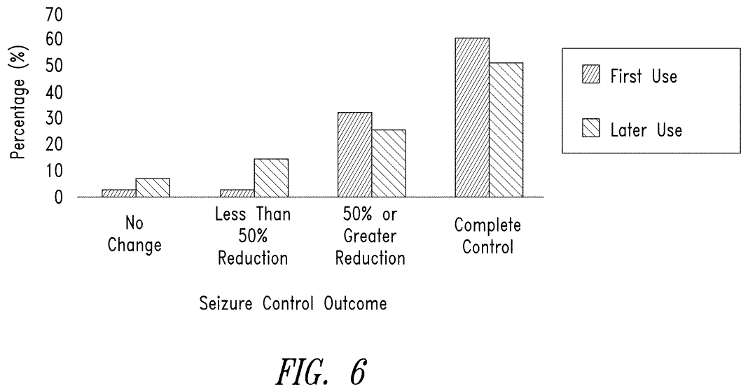

Last, seizure control outcome in individuals who used levetiracetam as the first AED was compared to seizure control outcome in individuals used levetiracetam later on in treatment. This comparison was done using the Cochran Armitage test, which yielded a p-value of 0.1877, which did not reach significance (see Table 12 and FIG. 6).

TABLE 12
Levetiracetam Used First vs. Later in Treatment

| Seizure control | | | |
|---|---|---|---|
| outcome for | First use | Later use | Trend |
| levetiracetam | (n, %) (n = 31) | (n, %) (n = 27) | test |
| No change | 1 (3.23) | 2 (7.41) | 0.1877 |
| Less than 50% reduction | 1 (3.23) | 4 (14.81) | |
| ≥50% reduction | 10 (32.26) | 7 (25.93) | |
| Complete control | 19 (61.29) | 14 (51.85) | |

Alternative Treatments

Only 3 individuals were reported to have used cannabidiol (CBD oil) and 5 were reported to have tried ketogenic diet. Because of the small n, statistical analysis was not done. Of the individuals reported to have used CBD oil, all 3 had used Charlotte's Web, a pure CBD oil preparation containing less than 0.3% THC. Response to Charlotte's Web improved seizure control in all 3 patients. Response to ketogenic diet was not as homogenous. Complete seizure control was achieved for 2 individuals on the diet. One individual was unable to maintain the diet long enough to evaluate seizure control, and no change in seizure activity was seen in the remaining 2 individuals.

### Discussion

The reported seizure history in this cohort was consistent with what has been published previously in the literature, making this a representative sample of the WHS population. The comparison of the five most commonly used AEDs showed no statistically significant difference in seizure control outcome. This indicates that levetiracetam and valproic acid provide the same level of control as diazepam, lamotrigine, and phenobarbital. There was also no statistical difference between starting with levetiracetam as the first AED and using it as a treatment later on. This implies that eventual seizure control outcome is not affected by whether the most effective treatment is tried first. In the comparison of carbamazepine to levetiracetam there is a statistically significant difference (p=0.0398) in seizure control outcome, demonstrating that carbamazepine is contraindicated in individuals with WHS.

This was the first study of its kind in the WHS population and has the largest cohort of individuals with WHS reported to date. This study provided statistical analysis ideas about seizure control in WHS previous postulated in the literature, but for which there was no formal study. The survey design also allowed for collection of AED history in chronological order, making it possible to compare the outcome of levetiracetam being used as a first seizure treatment to it being used later in treatment. Additionally, this study further demonstrates that the appropriate seizure treatment can vary with the genetic etiology, arguing for the importance of genetic testing at the onset of seizures to provide the best treatment right away and avoid treatments that can exacerbate seizures.

Although this study has many strengths, there are limitations that should also be noted. There is a possible selection bias; parents of children with WHS who are particularly motivated are more likely to complete the survey. Additionally, parents of children with a complicated seizure treatment history are more likely to leave the survey incomplete, due to concerns of accuracy and completeness. The lack of data about use of CBD oil may be related to the fact that PHI was collected in the survey. Parents living in states where use of CBD oil is not permitted may have been hesitant to report its use.

Levetiracetam and valproic acid are at least as effective as diazepam, lamotrigine, and phenobarbital in controlling seizures in WHS. Carbamazepine is less effective and has a much higher risk of exacerbating the number of seizures. Therefore, it is contraindicated in WHS. These results strongly support the importance of identifying the genetic etiology of seizures to guide treatment.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating Wolf-Hirschhorn syndrome (WHS) seizures comprising administering an effective amount of cannabidiol (CBD) to a subject identified as having a deletion of a seizure susceptibility region, wherein the seizure susceptibility region comprises 197 kbp starting 368 kbp from the terminal end of the short arm of chromosome 4.

2. The method of claim 1, wherein administering CBD reduces the frequency of seizures.

3. The method of claim 1, wherein the seizures are one or more of tonic-clonic seizures, clonic seizures, tonic spasms, myoclonic seizures, absence seizures, atonic seizures, complex partial seizures, simple partial seizures, atypical seizures, and status epilepticus.

4. The method of claim 1, wherein the deletion of the seizure susceptibility region was detected by chromosomal microarray.

5. The method of claim 1, wherein the subject has a diagnosis of WHS.

6. The method of claim 1, wherein the CBD is purified.

7. A method for reducing seizure activity comprising administering an effective amount of cannabidiol (CBD) to a subject identified as having a deletion of a seizure susceptibility region, wherein the seizure susceptibility region comprises 197 kbp starting 368 kbp from the terminal end of the short arm of chromosome 4.

8. The method of claim 7, wherein administering CBD reduces the frequency of seizures.

9. The method of claim 7, wherein the seizures are one or more of tonic-clonic seizures, clonic seizures, tonic spasms, myoclonic seizures, absence seizures, atonic seizures, complex partial seizures, simple partial seizures, atypical seizures, and status epilepticus.

10. The method of claim 7, wherein the deletion of the seizure susceptibility region was detected by chromosomal microarray.

11. The method of claim 7, wherein the subject has Wolf-Hirschhorn syndrome (WHS).

12. The method of claim 7, wherein the CBD is purified.

13. (canceled)

14. (canceled)

15. (canceled)

16. (canceled)

17. (canceled)

18. A method for reducing seizure activity comprising administering a combination of vitamin B6 and butyrate to a subject identified as having a deletion of a seizure susceptibility region, wherein the seizure susceptibility region comprises 197 kbp starting 368 kbp from the terminal end of the short arm of chromosome 4.

19. The method of claim 18, wherein administering the combination of vitamin B6 and butyrate reduces the frequency of seizures.

20. The method of claim 18, wherein the seizures are one or more of tonic-clonic seizures, clonic seizures, tonic spasms, myoclonic seizures, absence seizures, atonic seizures, complex partial seizures, simple partial seizures, atypical seizures, and status epilepticus.

21. The method of claim 18, wherein the deletion of the seizure susceptibility region was detected by chromosomal microarray.

22. The method of claim 18, wherein the subject has a diagnosis of Wolf-Hirschhorn syndrome (WHS).

23. The method of claim 22, comprising treating WHS seizures of the subject.
